(19) United States
(12) Patent Application Publication
(10) Pub. No.: US 2024/0353391 A1
(43) Pub. Date: Oct. 24, 2024

(54) SYSTEM FOR ASSESSING CELL VIABILITY, CELL MEMBRANE PIERCING AND SELECTING A CELL FOR MICROINJECTION

(71) Applicant: AGM MEDICAL, New York, NY (US)

(72) Inventor: Amir MOR, New York, NY (US)

(21) Appl. No.: 18/684,679

(22) PCT Filed: Aug. 18, 2022
(86) PCT No.: PCT/IB2022/057732

§ 371 Date: Feb. 18, 2024

(30) Foreign Application Priority Data
Aug. 18, 2021 (GB) 2111819.5

(51) Int. Cl. G01N 33/487; B01L 3/02; C12M 1/34

(52) U.S. Cl. CPC G01N 33/48728; B01L 3/021; C12M 41/46

(57) ABSTRACT

Detection apparatus for testing biological cells, including a measuring means for measuring an electrical property of a single biological cell present in a solution, sampling means for sampling the solution, a membrane of the cell, and/or an intracellular fluid of the cell, and an electrically insulating component located between the sampling means and the measuring means, containing therein an electrically conductive material. where the electrically insulating component has resistance to an alkaline solution of pH 8 to 11, where the resistance to the alkaline solution includes resistance to formation of microcracks for at least 1 minute upon exposure to the alkaline solution, resistance to electrical insulation failure for at least 12 minutes upon exposure to the alkaline solution, and resistance to visible structural integrity failure for at least 20 minutes upon exposure to the alkaline solution.

### FIELD OF THE INVENTION

The present invention provides an apparatus comprising a measuring means and a sampling means for use in selecting a biological cell according to an electrical property of the cell, wherein the apparatus has one or more alkaline-resistant components.

### BACKGROUND OF THE INVENTION

Determination of cell viability is important in many fields relating to cell biology and medicine, and there are several accepted assays of viability that utilize such diverse parameters as the redox potential of the cell population, the integrity of cell membranes, or the activity of cellular enzymes such as esterases. Each assay may or may not be suitable for the particular applications relating for cell viability, cytotoxicity, or drug efficacy with several integrated components. In the field of in vitro fertilization, the viability of eggs is typically assessed by visual inspection. The embryologist examines the zona pellucida, plasmatic membrane, and cell's cytoplasm. For example, abnormal cytoplasm appearance and/or fragmented plasmatic membrane (i.e., no evidence of intact smooth plasmatic membrane) are consistent with a non-viable cell/egg.

Intracytoplasmic sperm injection (ICSI) is an in vitro fertilization procedure in which a single sperm cell (spermatozoid) is injected directly into the cytoplasm of an oocyte (egg). This technique is used to obtain embryos that may be subsequently transferred to a maternal uterus. The ICSI procedure has been found to be an effective method of achieving fertilization and treating male factor infertility, although it may also be used where oocytes cannot be easily penetrated by sperm cells, and occasionally in addition to sperm donation. ICSI may be used, e.g., for males with teratospermia, whose sperm has abnormal morphology, and for males with azoospermia, whose semen contains no sperm cells.

In a typical ICSI procedure, oocyte viability is not considered prior to the injection. Sperm cells are injected into all available oocytes that have been aspirated from the ovaries. It is not certain at the time of injection which oocytes are viable, and which oocytes are dying. The practitioners count on the viable sperm cells to fertilize the viable eggs. When considering male partners with azoospermia, this lack of information becomes significant. These male partners usually undergo a testicular biopsy for sperm cell retrieval. If sperm cells are retrieved, their number is oftentimes low (a single digit number). When sperm cells number is the limiting factor for fertilization, the selection of the most viable eggs is critical. In these cases, injection of the few harvested sperm cells into the most viable eggs is the desired option.

Typically, during an intracytoplasmic sperm injection (ICSI) procedure, the egg is inspected by light microscopy only. However, it is generally known in the field that normal findings observed with light microscopy do not necessarily correlate with a viable egg. There are more accurate techniques to assess egg viability, but using them can lead to damage or complete destruction of the oocyte. Non-limiting examples of such techniques include electron microscopy, immunohistochemistry, and the usage of fluorescent dyes. There is a need for better technique capable of assessing cell viability while at the same time not damaging the cell (in the field of infertility and beyond).

Light microscopy has another limitation often encountered when attempting cell membrane penetration. During the ICSI procedure, the egg is penetrated with a sharp micropipette, which is used to inject a sperm cell directly into the egg. The actual cell penetration cannot always be clearly visualized by simple light microscopy. Therefore, the embryologist tries to aspirate cytoplasm contents into the pipette, prior to sperm injection, to confirm cell membrane penetration. This aspiration of cytoplasm into the pipette has the potential to damage the cell. Thus, this is an additional reason to seek for alternative techniques to confirm cell membrane penetration that lessens the likelihood of damage to the cell.

Nowadays, many women elect to have social egg freezing for various non-medical reasons. Egg freezing procedures for social reasons are usually paid out of pocket. Women usually choose to have 1-2 egg freezing cycles. Their eggs are frozen without any knowledge about the eggs' viability. In some cases, a significant number of eggs may be non-viable. In these cases, women may be falsely reassured that they have secured their fertility potential. The knowledge about how many eggs are actually viable and can be fertilized in the future around the time of egg retrieval can help the women and their provider decide whether additional egg retrieval cycles are needed in order to reliably secure the future fertility potential.

Embryologists who perform ICSI procedures relatively infrequently may not be comfortable with visual confirmation of sperm injection pipette advancement into the egg. There is a strong, unmet need in the art for an objective oocyte membrane penetration confirmatory method which is independent of the operator.

To date, robotic injection systems rely on complex computer vision algorithms and pressure sensing technologies in order to confirm pipette advancement into the cell. These methods still require the confirmation of cell membrane penetration by skilled human personnel. There is still a need for a highly reliable and reproducible technique confirming membrane penetration of a cell before attempting intracellular injection such as, e.g., a peptide, a protein, a fatty acid, a carbohydrate, a metal, a basic element, a nucleic acid (e.g., DNA, RNA), a vector (e.g., a viral vector), a microparticle (e.g., a virus, nanoparticle, liposome), a cell (e.g., a sperm cell), or a molecule (e.g., a small molecule, a dye, a fluorescent molecule, a reporter, a pharmaceutical). When a reliable technique (independent of human monitoring) is provided, the robotic injection systems can become fully automated. A method for assessing cell viability and cell membrane piercing is disclosed in PCT Publication No. WO 2020/077244, which is incorporated by reference as if fully set out herein.

### SUMMARY OF THE INVENTION

According to an aspect of some embodiments of the present invention, there is provided a detection apparatus for testing biological cells, the apparatus comprising a measuring means for measuring an electrical property of a single biological cell present in a solution; sampling means for sampling the solution, a membrane of the cell, and/or intracellular fluid of the cell; and an electrically insulating component located between said sampling means and said measuring means, containing therein an electrically conductive material, wherein said electrically insulating component has resistance to an alkaline solution of pH 8 to 11, wherein said resistance to said alkaline solution comprises at least one property selected from the group consisting of resistance to formation of microcracks for at least 1 minute upon exposure to said alkaline solution; resistance to electrical insulation failure for at least 12 minutes upon exposure to said alkaline solution; and resistance to visible structural integrity failure for at least 20 minutes upon exposure to said alkaline solution.

According to an embodiment, the electrical property is selected from a group consisting of (i) membrane potential, wherein the measuring means is an electrical potential meter; (ii) membrane resistance, wherein the measuring means is an electrical resistance meter; and (iii) membrane capacitance, wherein the measuring means an electrical capacitance meter, or combinations thereof.

According to an embodiment, the electrically conductive material is a metal and/or a conductive fluid.

According to an embodiment, the conductive material is silver coated with chloride. According to an embodiment, the sampling means comprising a micropipette tip or an electrical wire tip.

According to an embodiment, the detection apparatus is a cell injection system.

According to an embodiment, there is provided a system or kit comprising the detection apparatus as disclosed herein.

According to an embodiment, the system or kit further comprises a holding pipette. According to an embodiment, there is provided a method for selecting a cell for injection of a substance, comprising use of the detection apparatus as disclosed herein.

According to an embodiment, said selecting comprises selecting said cell for injection of a substance thereinto.

According to an embodiment, said detection apparatus is computerized and/or automated.

According to an embodiment, there is provided a method for selecting a viable cell, comprising use of the apparatus of claim 1.

According to an embodiment, there is provided a method of determining viability of a cell, the method comprising:
(a) providing a detection apparatus as disclosed herein, said sampling means comprising a tip, wherein said tip is configured to penetrate a membrane of the cell and wherein said measuring means comprises an electrical resistance meter connected directly or indirectly to said tip;
(b) measuring electrical resistance of said tip outside the cell;
(c) inserting said tip into the cell and measuring electrical resistance of said tip inside the cell;
(d) calculating resistance of the cell by subtracting the electrical resistance outside the cell of step (b) from the electrical resistance inside the cell of step (c); and
(e) determining viability of the cell by comparing the resistance of the cell of step (d) with a control.

According to an embodiment, there is provided a method for identifying piercing of a membrane of a cell by use of the detection apparatus as disclosed herein.

According to an embodiment, there is provided a method for identifying piercing of a membrane of a cell, the method comprising:
(a) providing a detection apparatus as disclosed herein, said sampling means comprising a tip, wherein said tip is configured to penetrate a membrane of the cell and wherein said measuring means comprises an electrical resistance meter connected directly or indirectly to said tip;
(b) measuring electrical resistance of said tip outside the cell;
(c) advancing said tip towards the cell while measuring electrical resistance of said tip; and
(d) determining membrane piercing by said tip by a detected increase in resistance.

### BRIEF DESCRIPTION OF THE DRAWINGS

FIG. 1 (prior art) depicts a schematic diagram of a system for selecting biological cell.

FIG. 2 (prior art) depicts a photograph of a non-limiting example of a multimeter suitable for measuring resistance.6

FIG. 3 (prior art) schematically illustrates an exemplary method of measuring membrane resistance using an ICSI pipette of the system as described herein.

FIG. 4 (prior art) shows absolute resistances obtained using the exemplary method of ICSI pipette-based measurement.

FIGS. (prior art) 5A and 5B show ROC analysis of the change in resistance (ΔR) in the fragmented/ruptured membrane group (negative control group, n=12) vs. ΔR in the positive plasmatic membrane piercing group (determined by visualization only, study group, n=45).

FIG. 6 (prior art) depicts an egg with visually intact membrane.

FIG. 7 (prior art) depicts an egg with fragmented membrane.

FIGS. 8A and 8B (prior art) illustrate zona pellucida penetration by the ICSI pipette tip of the system as disclosed herein but no plasmatic membrane penetration; before (FIG. 8A) and after (FIG. 8B) positive pressure application through the pipette (negative control group, n=7).

FIGS. 9A to 9P (prior art) show screenshots from a video documenting zona pellucida penetration by the ICSI pipette tip of the system as disclosed herein but no plasmatic membrane penetration before or after positive pressure application through the pipette (negative control group).

FIGS. 10A to 10L (prior art) depict screenshots from a video documenting zona pellucida and plasmatic membrane penetration by the ICSI pipette tip of the system as disclosed herein as well as membrane rupture following the application of positive pressure through the pipette (positive control group).

FIGS. 11A to 11F (prior art) depict characterization of electrical resistance when piercing versus not piercing the oolemma. The resistance is about 9 MΩ before penetrating the oocyte, (FIG. 11A). The resistance increases to 14 MΩ after penetrating the oocyte, (FIG. 11B). The application of positive pressure through the ICSI pipette tip leads to oolemma rupture and, in return, the resistance decreases back to about 9 MΩ, (FIG. 11C). In contrast to the sequence depicting in FIGS. 11A-11C, the sequence depicted in FIGS. 11D-11E (different oocyte) demonstrates resistance measurements when the pipette tip has never penetrated the oocyte. In this case the resistance does not increase (remains stable around 9 MΩ). Positive pressure application confirms oocyte non-penetration by distending the zona pellucida while not rupturing the oocyte, (FIG. 11E). The summary of the proof of concept experiments shows a significant resistance increase in the Penetrated group only, (FIG. 11F).

FIG. 12 (prior art) depicts electrical resistance measurements in oocytes with intact oolemma vs. fragmented mouse oocytes. Fragmented oocytes are considered non-viable. In this experiment, the fragmented mouse oocytes showed a resistance increase of up to 2.2 MΩ. Therefore, intact mouse oocytes showing resistance increase equal or less than 2.2 MΩ are likely to be non-viable. Intact mouse oocytes showing resistance increase of more than 2.2 MΩ are considered viable.

FIG. 13 (prior art) illustrates exemplary measurement of electrical resistance change in human oocytes using a commercial ICSI system. There is a significant resistance increase when penetrating the oocyte with intact oolemma (top picture). Of note, leaning against the oolemma does not result in a significant resistance increase.

FIG. 14 (prior art) depicts electrical resistance measurements in oocytes with intact oolemma vs. fragmented human oocytes. Fragmented oocytes are considered non-viable. In this experiment, the fragmented human oocytes showed a resistance increase of up to 0.3 MΩ. Therefore, intact human oocytes showing resistance increase equal or less than 0.3 MΩ are likely to be non-viable. Intact human oocytes showing resistance increase of more than 0.3 MΩ are considered viable. AUC, area under the curve. CI, confidence interval.

FIG. 15A (prior art) depicts an exemplary adaptor for holding a pipette as contemplated herein. FIG. 15B depicts a zoomed-in view of the adaptor highlighting a wire connected to the electrode wire on one end and connected to an alligator clip of a cable that can connect to one or more measuring devices.

FIGS. 16A and 16B (prior art) depict cross-sectional drawings of an exemplary adaptor as contemplated herein.

FIG. 17 (prior art) illustrate a comparison of the concept of evaluating the viability a cell using electrical resistance versus using light microscopy.

FIG. 18 schematically illustrates a detection apparatus in accordance with the principles of the present invention.

FIG. 19 schematically illustrates an exemplary sampling means as part of the apparatus as contemplated herein.

FIG. 20 schematically illustrates an exemplary adaptor as contemplated herein.

FIG. 21 schematically illustrates an alternative embodiment of an adaptor as contemplated herein

FIG. 22 schematically illustrates an alternative embodiment of a sampling means as contemplated herein

FIG. 23 schematically illustrates the embodiment of a sampling means of FIG. 22, further comprising a micropipette head grip.

FIG. 24 schematically illustrates presence of chloride debris that clog the micropipette's tip and interfere with electoral readings as well as fluid flow through the tip.

FIG. 25 is a line graph showing the effect of exposure to bleach on polycarbonate adapter.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In one embodiment, the subject is a human.

As used herein, the term “cell” refers to any eukaryotic cell, including mammalian cells, avian cells, amphibian cells, plant cells, fish cells, and insect cells, whether located in vitro or in vivo. As used herein, the term “cell culture” refers to any in vitro population of cells. Included within this term are continuous cell lines (e.g., with an immortal phenotype), primary cell cultures, transformed cell lines, finite cell lines (e.g., non-transformed cells), and any other cell population maintained in vitro.

The term “intracytoplasmic sperm injection” or “ICSI” refers to an in vitro fertilization procedure in which a single sperm is injected or microinjected directly into an oocyte. This procedure is most commonly used to overcome male infertility factors, although it may also be used where oocytes cannot easily be penetrated by sperm, and occasionally as a method of in vitro fertilization, especially that associated with sperm donation.

The term “oocyte”, “egg cell”, and “egg” refers to a female gametocyte or germ cell involved in reproduction. In other words, it is an immature or mature ovum, or egg cell. An oocyte is produced in the ovary during female gametogenesis. In some embodiments, oocytes for use in the invention are mammalian, including but not limited to human, livestock (including but not limited to bovine, porcine, and ovine) and companion animal (including but not limited to canine and feline).

The terms “sperm”, “sperm cell”, “spermatozoon”, and “spermatozoid” are used interchangeably herein to refer to a male gametocyte or germ cell involved in reproduction. A sperm cell is produced in the testis during male gametogenesis. In some embodiments, sperm for use in the invention are mammalian, including but not limited to human, livestock (including but not limited to bovine, porcine, and ovine) and companion animal (including but not limited to canine and feline).

The terms “membrane potential”, “transmembrane potential”, and “membrane voltage” are used interchangeably herein to refer to the difference in electric potential between the interior and the exterior of a biological cell.

The term “electrical conductor” refers to an object or a type of material that allows the flow of an electrical charge (such as an electron, a proton, and/or an ion) in one or more directions. Non-limiting examples of electrical conductors include metal (e.g., copper, gold, silver), non-metal material (such as graphite), and fluid (e.g., a liquid or a gel) containing charged particles (e.g., electrolytes).

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### Electrophysiological Measurements

Electric potential across the cell membrane is a common feature of living cells. Its magnitude depends on the type as well as the physiologic status of the cell. Different cell types can have different membrane potentials. Cells of a certain type (e.g., oocytes) of different species also can have different membrane potentials. Further, certain cells can have different membrane potentials at different stages in development. For example, oocytes can have different membrane potentials before and after fertilization, as well as at first cleavage (see, e.g., U.S. Pat. No. 6,927,049; Tyler et al., Biological Bulletin, 1965, 111(1): 153-177; Morrill et al., J. Cell Physiol., 1965, 67:85-92).

Membrane potential is the difference in electric potential between the interior and the exterior of a biological cell. With respect to the exterior of the cell, typical values of membrane potential range from −10 mV to −80 mV. All animal cells are surrounded by a membrane composed of a lipid bilayer with proteins embedded in it. The membrane serves as both an insulator and a diffusion barrier to the movement of ions, including positively and negatively charged ions (e.g., K⁺, Na^{+,} Ca²⁺, Cl⁻) via transmembrane proteins such as ion channels and ion transporters. Ion transporter or ion pump proteins actively move ions across the membrane to establish ion concentration gradients across the membrane, while ion channels allow ions to move across the membrane down those concentration gradients. Active ion pumps and passive ion channels can be thought of as equivalent to a set of batteries and resistors inserted in the membrane, and therefore create a voltage between the two sides of the membrane. Generally, eukaryotic cells (including cells from animals, plants, and fungi) maintain a non-zero transmembrane potential, usually with a negative voltage in the cell interior as compared to the cell exterior. The membrane potential can enable a cell to function as a battery, providing power to operate a variety of “molecular devices” embedded in the membrane. Also, in electrically excitable cells such as neurons and muscle cells, the membrane potential can also enable transmitting signals between different parts of a cell. Signals are generated by opening or closing of ion channels at one point in the membrane, producing a local change in the membrane potential. In non-excitable cells, and in excitable cells in their baseline states, the membrane potential is held at a relatively stable value, called the resting potential.

In electrically active tissue, the difference in potential between any two points can be measured by inserting an electrode at each point, for example one inside and one outside the cell, and connecting both electrodes to the leads of a voltmeter, an electrical resistance meter, and/or an electrical capacitance meter. Generally, the zero potential value is assigned to the outside of the cell and the sign of the potential difference between the outside and the inside is determined by the potential of the inside relative to the outside zero.

The membrane potential in a cell derives from at least two factors: electrical force and diffusion. Electrical force arises from the mutual attraction between particles with opposite electrical charges (positive and negative) and the mutual repulsion between particles with the same type of charge (both positive or both negative). Diffusion arises from the tendency of particles to redistribute from regions where they are highly concentrated to regions where the concentration is low. Both of these factors influence the movement of ions across the cell membrane, which leads to the generation of electrical signals.

Because cell membranes are made of lipid bilayers, the plasma membrane intrinsically has a high electrical resistivity, in other words a low intrinsic permeability to ions. This low permeability is countered by the presence of transmembrane proteins that either actively transport ions from one side of the membrane to the other or provide channels through which they can move or diffuse.

In electrical terminology, the plasma membrane functions as a combined resistor and capacitor (see, e.g., Rettinger J., Schwarz S., Schwarz W. (2016) Basics: Theory. In: Electrophysiology, Basics, Modern Approaches and Applications. Springer International Publishing). Because the membrane impedes the movement of ions across it, it can be considered a resistor. The thinness of the lipid bilayer (about 7-8 nanometers) enables an accumulation of charged particles on one side of the membrane, which gives rise to an electrical force that pulls oppositely charged particles toward the other side, and thus provides the capacitance. The capacitance of the membrane is relatively unaffected by the molecules that are embedded in it, so it has a more or less invariant value. The conductance of a pure lipid bilayer is generally very low, so that it is generally dominated by the conductance of alternative pathways provided by the transmembrane proteins. Thus, the capacitance of the membrane is more or less fixed, but the resistance is highly variable.

A cell's resistance is a measure of how easily ions can move through the membrane. Generally, the fewer channels there are for ions to flow through, the higher the resistance of the cell will be. In other words, the resistance of a lipid bilayer membrane to the passage of ions across it is very high, but transmembrane proteins can greatly enhance ion movement, either actively or passively, via facilitated transport and facilitated diffusion, respectively. Ion channels provide passageways through which ions can passively move (i.e., facilitated diffusion). Generally, an ion channel is permeable only to specific types of ions (e.g., sodium and potassium but not chloride or calcium), and the permeability can vary depending on the ion's concentration gradient. Ion channel proteins have different configurations that open and close the channel (also called a pore) and can change conformation based on voltage changes across the membrane, binding of a ligand to the channel proteins, and in response to various stimuli (e.g., heat, light). Ion pumps, also known as ion transporters or carrier proteins, actively transport specific types of ions from one side of the membrane to the other, sometimes using cellular energy (e.g., ATP) to do so, and thus can move ions against their concentration gradient. Common ion pumps include sodium-potassium pumps and sodium-calcium exchangers.

A cell's capacitance determines how quickly the membrane potential can respond to a change in current. A capacitor is made up of two conducting materials separated by an insulator. In the case of a cell, the extracellular and intracellular fluids are the conductors, and the lipid bilayer membrane is the insulator. When there is a voltage difference (such as the resting membrane potential) across an insulator, charge will build up at the interface because current cannot flow directly across the insulator. The constant that describes the relationship between the voltage and the charge that builds up is called the capacitance. When this built-up charge becomes large enough, an induced (capacitive) current is produced, which can change the membrane voltage. As the membrane voltage increases, the ion pumps and channels can open to allow charge to move across the membrane.

The capacitance of the cell membrane is high because it is only two molecules (phospholipids) thick, meaning that not much voltage is needed to separate charges across the membrane. The specific capacitance of biological membranes is very close to what is obtained simply from the dielectric constant of lipids and the thickness of the and, unlike the conductance, the capacitance of a cell membrane is generally constant. Further, the membrane capacitance can be measured in terms of the area of the membrane, such that the larger the area, the larger the capacitance.

The electrical properties of cell membranes can be measured by, e.g., electrodes (including arrays of electrodes), and pipettes (see, e.g., Rettinger J., Schwarz S., Schwarz W. (2016) Basics: Theory. In: Electrophysiology, Basics, Modern Approaches and Applications. Springer International Publishing; Narahashi T, Principles of electrophysiology: an overview. Curr Protoc Toxicol., November 2003, Chapter 11).

It has long been thought that pipettes suitable for use in measuring membrane potential must be of a small diameter, in order to avoid significantly disrupting the cell membrane and intracellular environment and thus damaging the cell. These pipettes can have a relatively small bore diameter that is about 1 micrometer to about 2.5 micrometers.

Further, larger bore pipettes are thought to be too large for the cell membrane to form a seal around the pipette and enable accurate measurement of the membrane potential (Polcz et al., Fertility and Sterility, 1997, 68(4) 735-738). Some reasons leading to unreliable measurement of membrane potential with large bore pipettes (such as for example and not limitation, ICSI pipettes) include:
1. Larger leak current around the pipette's wall as described herein. This leak current can be too large for the cell to compensate for. The result is usually a forced depolarization of the resting membrane potential such that it is very difficult to measure the true physiological negative value of the membrane potential. This forced depolarization of the resting membrane potential can be falsely interpreted as a dying/dead cell.
2. Use of a large bore pipette can enable rapid mixture of the electrolyte in the pipette with the cell's cytoplasm. During the ICSI procedure, the sperm is aspirated into the pipette with extracellular solution. Immediately after cell membrane piercing, there is generally a rapid mixing of the solution in the pipette with the intracellular solution. This mixing can lead to a rapid decrease of ionic concentration gradient across the membrane that in turn can depolarize the resting membrane potential towards 0 mV or can even lead to its forced cancellation. In other words, as long as the ICSI pipette is in the cell, the cell may not be able to fully correct the massive disruption of the intercellular ionic concentrations, and therefore the resting membrane potential may be forced to depolarize and even cancel completely. Following sperm injection and the retraction of the ICSI pipette, the majority of the eggs recover and regain their negative physiological resting membrane potential. While small bore pipettes can inhibit the rapid mixture of solutions, they generally are not suitable for sperm injection due to their small tip diameter. Additionally, the electrolyte solution used in small bore regular electrophysiological pipettes generally contains ionic concentrations similar to those of the intracellular environment rather than the extracellular environment, and therefore can lead to less disruption of the intracellular environment. Additionally, large bore pipettes together with hardware limitations lead to inevitable continuous fluxionalities in voltage, capacitance and/or resistance measurements. Therefore, physiological changes of voltage, capacitance, and/or resistance may be overlooked simply because they fall within the ‘noise’ range (using large bore pipettes).

Due to the reasons above, it is generally accepted by persons skilled in the art that, as for voltage, membrane resistance and capacitance measurements are not feasible using a relatively large bore pipette containing an electrolyte solution similar to the extracellular environment.

Surprisingly, the inventors have found that, while large bore pipettes can interfere with accurate resting membrane potential, there is a measuring technique using large bore pipettes, such as, for example and not limitation, those used in ICSI to deliver sperm cells to the oocyte, which does not significantly disrupt measurements of membrane resistance and/or capacitance. Accordingly, in certain embodiments, the present invention provides methods for measuring cell resistance using one or more large bore pipettes such as ICSI pipettes. The methods of the present invention produce a clear, reproducible, and stable increase in measured resistance upon piercing of a cell membrane using these methods. These pipettes can have bore diameters that are about 4 micrometers to about 10 micrometers, or about 4 micrometers to about 7 micrometers, or about 4 micrometers to about 6 micrometers, or about 4 micrometers, or about 5 micrometers, or about 6 micrometers. The pipettes can be made of any suitable material as understood in the art, including for example, one or more biocompatible plastics such as polycarbonate and/or polyvinyl, glass, and the like. The pipette may include one or more pipettes having an electrical wire in the pipette lumen and/or one or more pipettes having one or more conductive material adhered to the inner pipette wall (e.g., glass pipette-carbon fiber).

The large bore pipettes also enable real-time measurement of the membrane resistance and/or capacitance to enable real-time determinations of viability and membrane piercing.

### Penetration of Cell Membrane

Intracytoplasmic sperm injection (ICSI) has been found to be an effective method of achieving fertilization and treating male factor infertility. The progress of the ICSI technique, however, has been dependent primarily on trial and error strategies, with success rates heavily dependent on the experience of the practitioner performing the procedure (Polcz, T.E. et al., Fertility and Sterility, 1997, 68, 4, 735-738; Neri et al., Cell Calcium, 2014 January; 55(1), 24-37).

Of the various steps in the process, penetration of the cell membrane of the oocyte presents the greatest difficulty. One technique to facilitate and confirm penetration is to aspirate ooplasm into the pipette before injecting the sperm cell into the egg. This method may be disadvantageous in that it may disrupt cell structures such as the cytoskeleton, spindles, or genetic material.

One method of confirming oocyte membrane penetration involves measuring the electrical potential of the membrane with an electrode through a small bore pipette. However, in the context of ICSI, incorporating an electrophysiological pipette-based confirmation of membrane penetration adds another step, as the pipette for confirming penetration is of too small a diameter to inject a sperm cell or spermatozoon. As described above, the general knowledge in the field suggests that the diameter of pipettes used to determine membrane potential must be small in order to allow an accurate measurement, and also to prevent intracellular contents from leaking out.

Once the cell membrane has been pierced, the use of a large bore pipette enables a wide variety of materials to be introduced into the cell. Non-limiting examples of such materials include a peptide, a protein, a fatty acid, a carbohydrate, a metal, a basic element, a nucleic acid (e.g., DNA, RNA), a vector (e.g., a viral vector), a microparticle (e.g., a virus, nanoparticle, liposome), a cell (e.g., a sperm cell), or a molecule (e.g., a small molecule, a dye, a fluorescent molecule, a reporter, a pharmaceutical).

In certain embodiments, the present invention provides methods for determining oocyte viability. The methods include first penetrating the membrane of a cell using one or more ICSI pipettes as described herein.

In certain embodiments the methods further include measuring the resistance of the cell. The resistance can be measured using standard techniques as understood in the art, including using a standard multi-meter capable of measuring resistance.

Embodiments of the methods further include analyzing the measured resistance by comparing the measured resistance value to a threshold control resistance. In certain embodiments the threshold control is about 0.5 MΩ to about 1 MΩ, about 1 MΩ to about 1.5 MΩ, about 1.5 MΩ to about 2 MΩ, abot 2 MΩ to about 2.5 MΩ, about 2.5 MΩ to about 3 MΩ. About 3 MΩ to about 3.5 MΩ. About 3.5 MΩ to about 4 MΩ and so on. In certain embodiments, the threshold resistance is about 0.9 MΩ. In certain embodiments, the threshold resistance is about 2.2 MΩ.

Embodiments of the methods further include determining cell viability of the measured cell. In certain embodiments, the cell is determined to be viable if the measured resistance is greater than the threshold resistance. In certain embodiments, the cell is determined to the viable if the measured change in resistance (ΔR) exceeds the threshold resistance by a non-zero value (i.e. has a difference in resistance ΔR) of between about 0.001 MΩ and about 0.01 MΩ, about 0.1 MΩ and about 0.5 MΩ, about 0.5 MΩ and about 1 MΩ, about 1 MΩ and about 1.5 MΩ, about 1.5 MΩ and about 2.0 MΩ, about 2.0 MΩ and about 2.5 MΩ, about 2.5 MΩ and about 3.0 MΩ, about 3.0 MΩ and about 3.5 MΩ, about 3.5 MΩ to about 4.0 MΩ, about 4.0 MΩ to about 4.5 MΩ, about 4.5 MΩ to about 5.0 MΩ, about 5.0 MΩ to about 5.5 MΩ, about 5.5 MQ to about 6.0 MΩ, about 6.0 MΩ to about 6.5 MΩ, about 6.5 MΩ to about 7.0 MΩ, about 7.0 MΩ to about 7.5 MΩ, about 7.5 MΩ to about 8.0 MΩ, about 8.0 MΩ, to about 8.5 MΩ, about 8.5 MΩ to about 9.0 MΩ, about 9.0 MΩ to about 9.5 MΩ, about 9.5 MΩ to about 10 MΩ, greater than about 10 MΩ, including values therebetween. In certain instances, an intact, viable cell may have a change in resistance value that is low (e.g., below a threshold value) and appear visually intake. This may indicate that the cell has not been sufficiently punctured or penetrated, that the cell is in fact non-viable, and/or that there is a large leak current.

The method may further include injecting the cell if the cell is determined to be viable, and not injecting the cell if the cell is determined to be not viable. If the cell is determined to be viable, the cell may be injected with one or more materials including a peptide, a protein, a fatty acid, a carbohydrate, a metal, a basic element, a nucleic acid (e.g., DNA, RNA), a vector (e.g., a viral vector), a microparticle (e.g., a virus, nanoparticle, liposome), a cell (e.g., a sperm cell), or a molecule (e.g., a small molecule, a dye, a fluorescent molecule, a reporter, a pharmaceutical), as contemplated herein.

Embodiments of the methods may further include measuring resistance as contemplated herein in order to confirm that an embryo has been successfully deployed from a transfer catheter.

Embodiments of the methods may include measuring resistance as contemplated herein in order to confirm successful lumber puncture (LP) and intravenous (IV) access.

Embodiments of the methods may include measuring resistance of Xenopus laevis eggs using the methods as contemplated herein. Measurement of a first resistance increase may be used to indicate piercing of the plasmatic membrane of Xenopus laevis eggs. Measurement of a second resistance increase may be used to indicate piercing the nuclear envelope.

### Automation of Membrane Resistance Measurement and Cell Membrane Penetration

In certain embodiments, the methods of the invention can be performed by a human or can be automated such that a robotic system can perform them. Non-limiting exemplary automated systems are described in WO 2008/034249; WO 2012/037642; WO 2013/158658; Huang et al., IEEE Transactions on Robotics, 2009, 25(3), 727-737; Lu et al., IEEE Transactions on Biomedical Engineering, 2011, 58 (7), 2102-2108; Karimirad et al., 23rd IEEE International Symposium on Robot and Human Interactive Communication, August 2014, 347-352.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Egg Retrieval

Female mice were injected with Follicular Stimulating Hormone (FSH) and human Chorionic Gonadotropin (hCG) in order to mature their eggs. The mice were sacrificed and their ovaries and fallopian tubes were harvested. The eggs were separated from the ovaries and tubes under the microscope. The eggs were transferred into commercial MII medium (water based solution) in order to keep them viable.

### Example 2: Cell Setup for Electrophysiological Measurements

A droplet of commercial MII medium was placed on a petri dish. Mineral oil was added until it fully covered the droplet to prevent droplet evaporation.

A single egg was placed in the droplet using a commercial transfer pipette. The petri dish with the egg was placed under the microscope of the electrophysiological setup. Two robotic micromanipulators were used:

A commercial egg/cell glass holding pipette with a tip curved at 30 degrees was attached to the left micromanipulator. A commercial egg/cell glass injection pipette with a tip curved at 30 degrees was attached to the right manipulator. Of note, the injection pipette was prefilled with MII medium and was attached to an electrophysiological headstage (available, e.g., from Axon instruments). A silver wire originating from the headstage was in contact with the MII medium in the injection pipette.

The headstage was connected to a standard amplifier (available, e.g., from Axon instruments), which was fully controlled by a computer software.

The egg was attached to the holding pipette by applying vacuum.

An electrical ground wire (electrode) was placed into the petri dish liquid (MII medium droplet surrounded by oil). Additional MII medium is added to the petri dish in order to create a continuous aqueous extracellular solution between the egg and the ground electrode.

The setup was thus complete, with the cell ready for electrical resistance measurements.

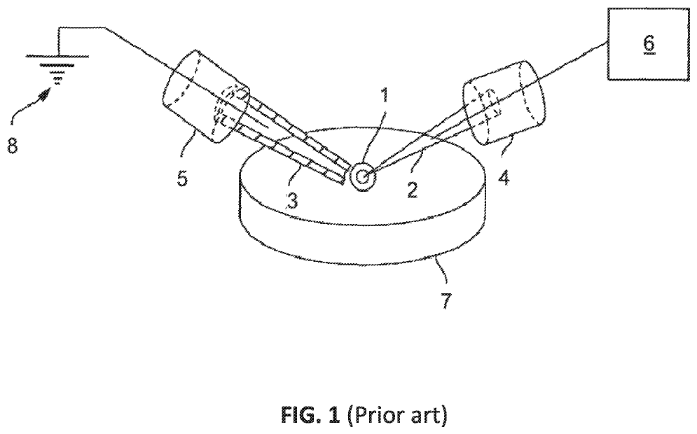

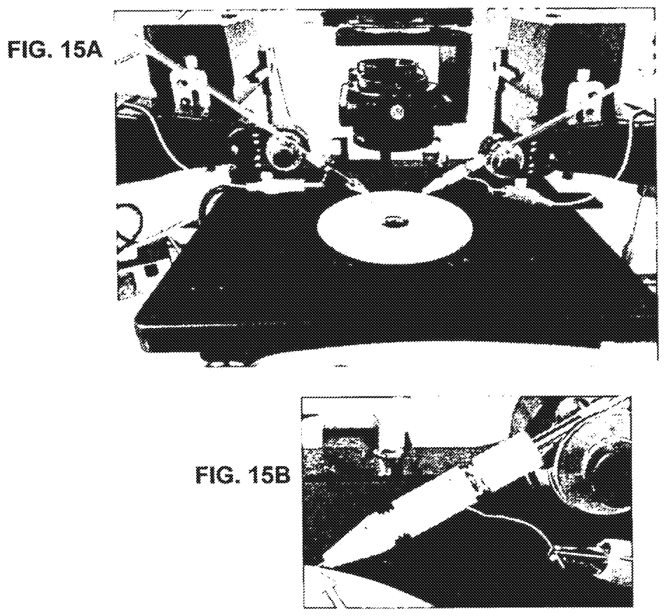

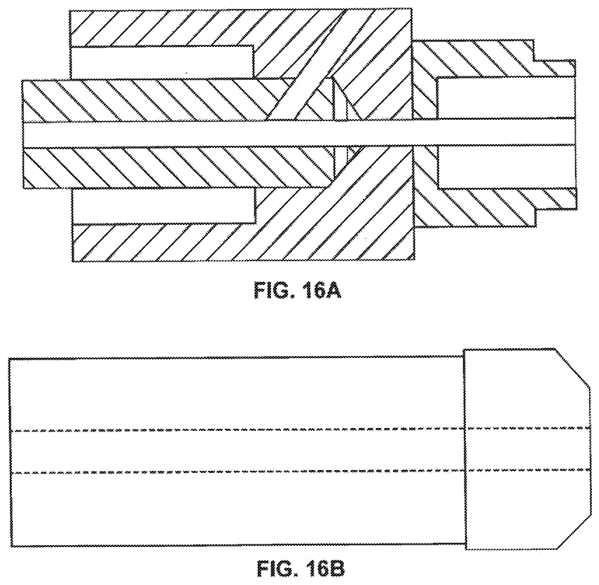

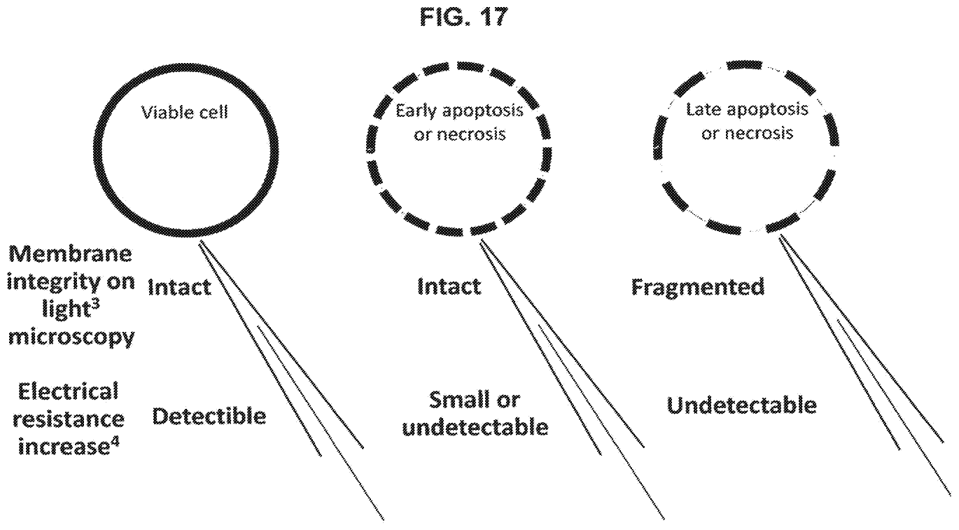

An exemplary cell setup according to WO 2020/077244 is depicted in FIG. 1. In this setup, a cell (1) is placed in a Petri dish (7) with extracellular solution. The cell (1) is held in place with a holding micropipette (3) which is coupled to an electrical ground connection (8). Material can be injected into the cell (1) via an injection micropipette (2) that is coupled to an electrophysiological amplifier (6). Each of the holding micropipette and the injection micropipette are also coupled to air or oil microinjectors that apply a controlled vacuum or pressure. The injection pipette may be integrated with one or more adaptors (FIGS. 15A and 15B) for ease of handling.

### Example 3: Electrophysiological Measurements

Electrical resistance of a cell was measured in WO 2020/077244 using an injection pipette according to the following procedure.

A short test pulse of 10 mV was generated by the headstage and amplifier. The generated electrical current is measured through the tip of the glass injection pipette (by the amplifier). The injection pipette tip resistance was calculated according to Ohm's law (V=IR). Every 0.5 second a 10 mV test pulse was given for 50 milliseconds. This allows for continuous rechecking of the injection pipette resistance.

The injection pipette resistance was initially measured in the MII solution outside the egg. Then, the injection pipette was advanced into the egg. The pipette resistance was measured while its tip was in the egg. Then, the pipette was withdrawn and the resistance was again measured when its tip was outside the egg (this was done in order to demonstrate effect reversal).

The egg's resistance is calculated according to the following:

Egg resistance=(pipette resistance when its tip is inside the egg)−(pipette resistance when its tip is outside the egg).

At the end, eggs were incubated with sperm cells and monitored for fertilization.

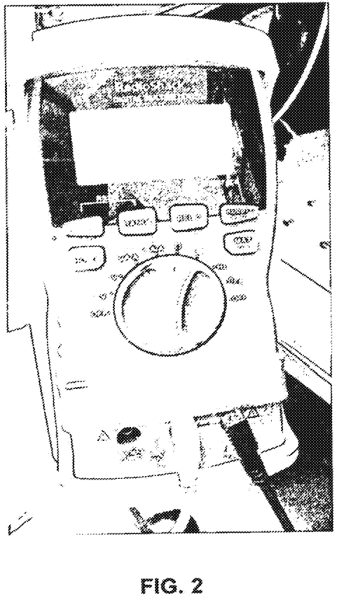

An exemplary multimeter according to WO 2020/077244, which can measure resistance and capacitance, is shown in FIG. 2.

### Example 4: Determination of Cell Membrane Piercing

The ICSI pipette-based method of determining cell membrane piercing via measurement of membrane electrophysiology described herein was tested in WO 2020/077244 in comparison with a known visual method of determining cell membrane piercing via light microscopy.

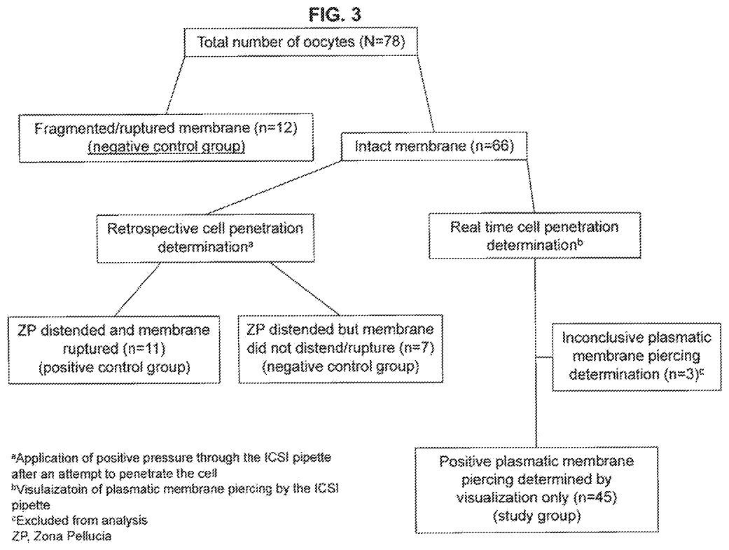

The ICSI pipette-based electrophysiology testing was carried out according to the schematic shown in FIG. 3.

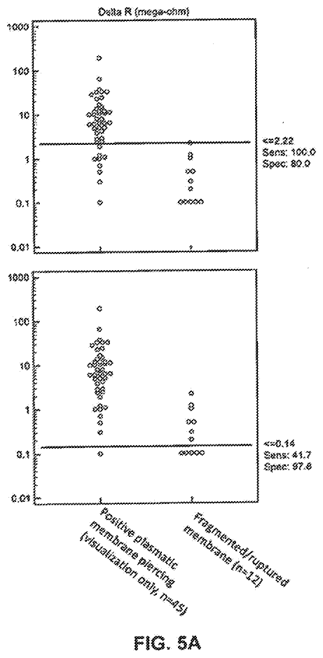

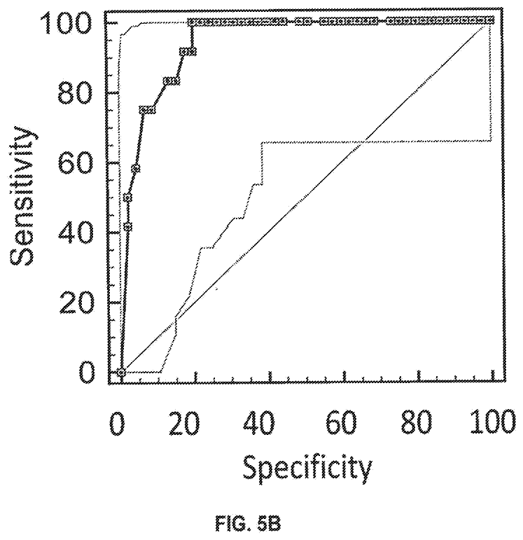

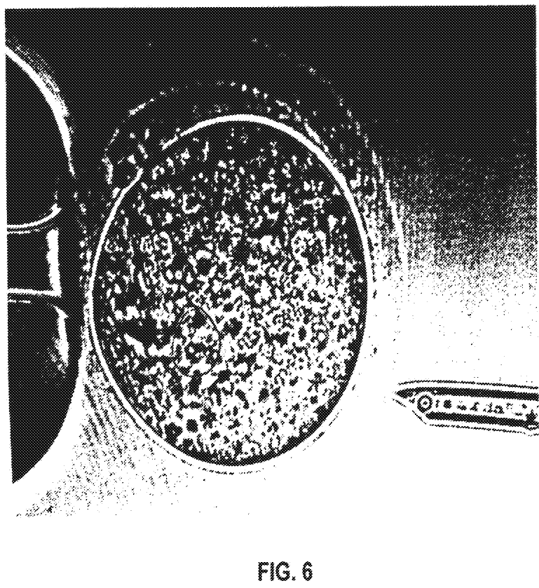

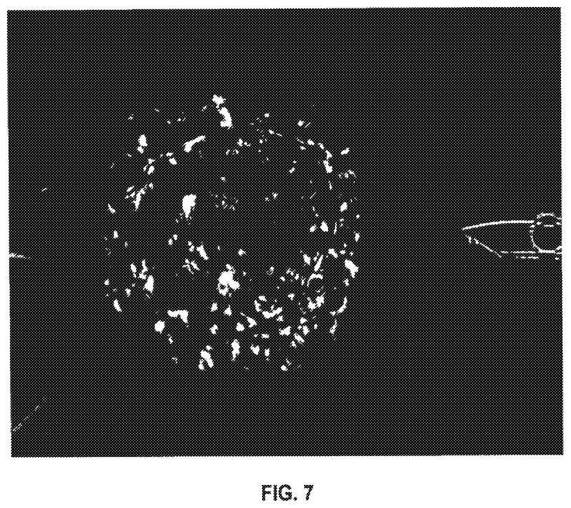

Using an ICSI pipette to measure membrane electrophysiology enabled the determination of both cell membrane piercing and cell membrane integrity (which is indicative of the cell's viability; an oocyte with a visually intact membrane is shown in FIG. 6 while a cell with a visually fragmented membrane is shown in FIG. 7). When the ICSI pipette was advanced into a cell, the resistance across the pipette tip increased. It was found that the higher the change in resistance (ΔR), the more resistant/intact the membrane was.

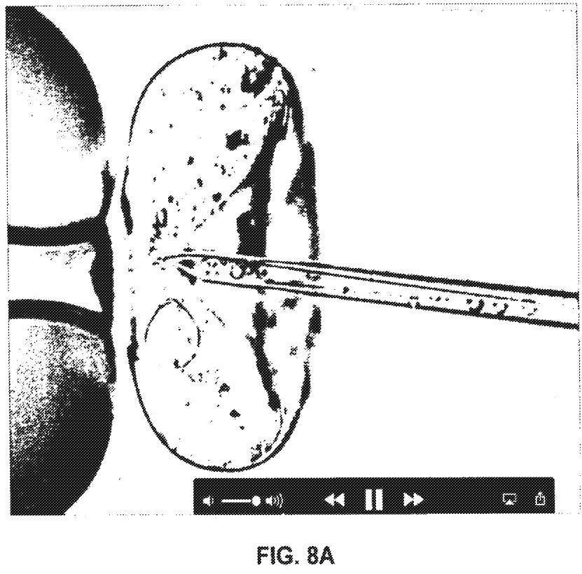

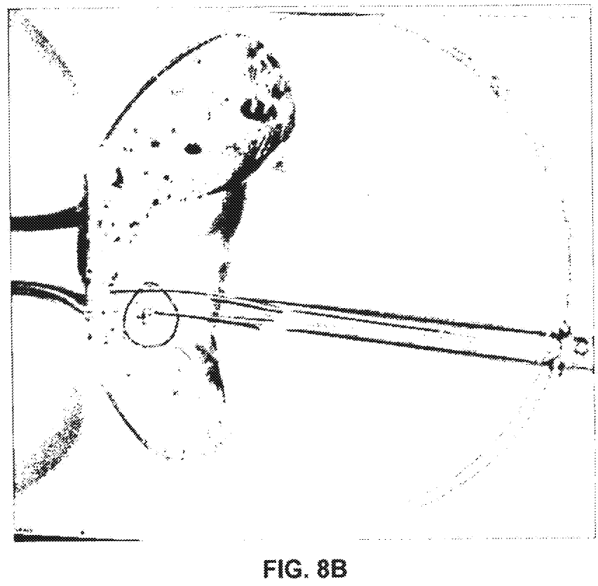

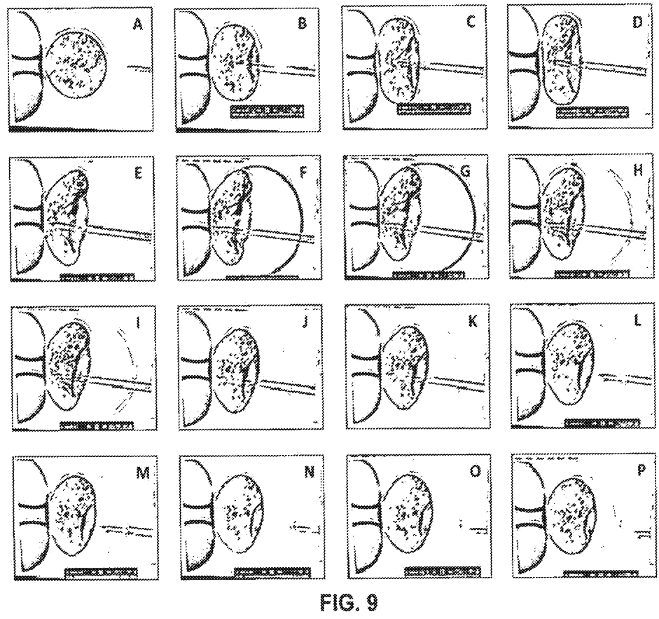

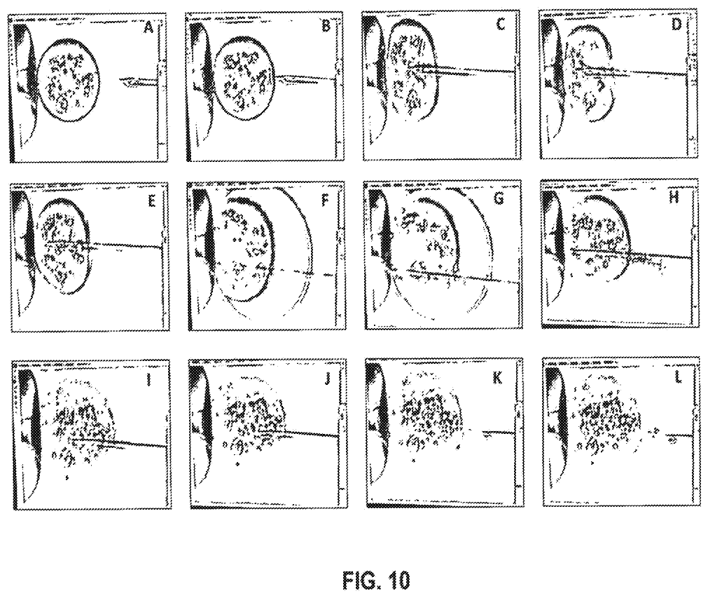

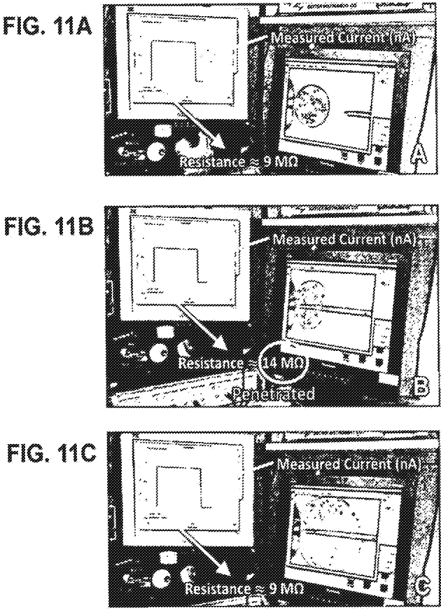

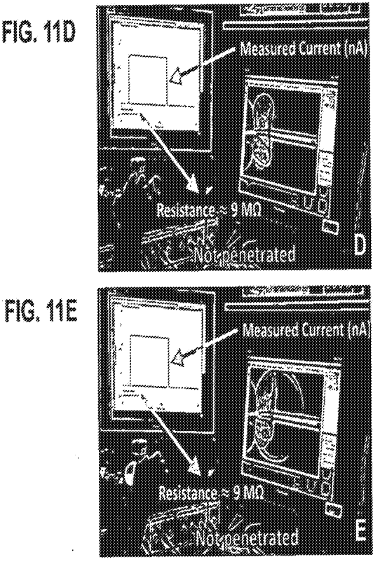

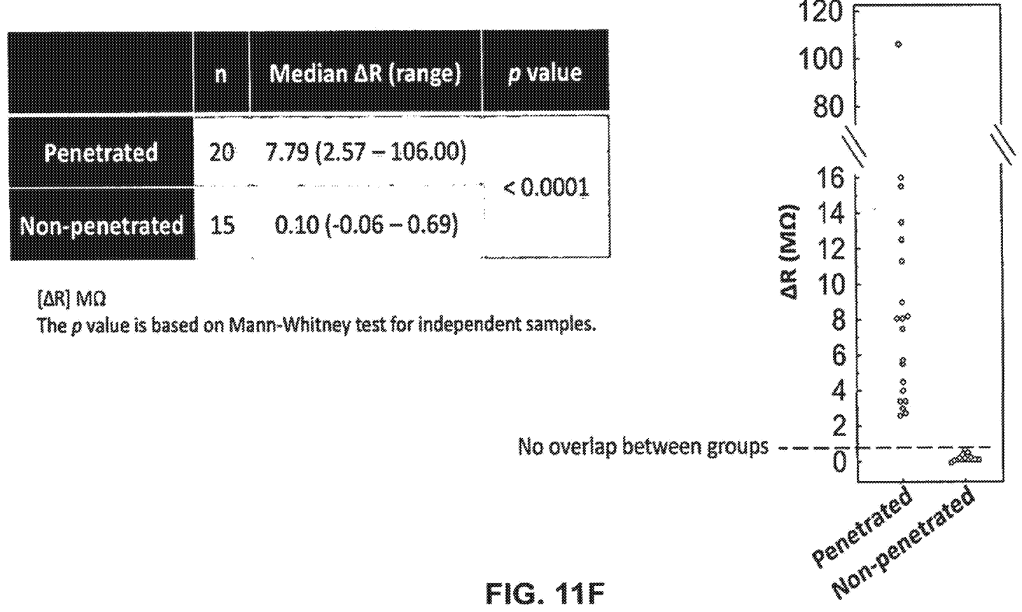

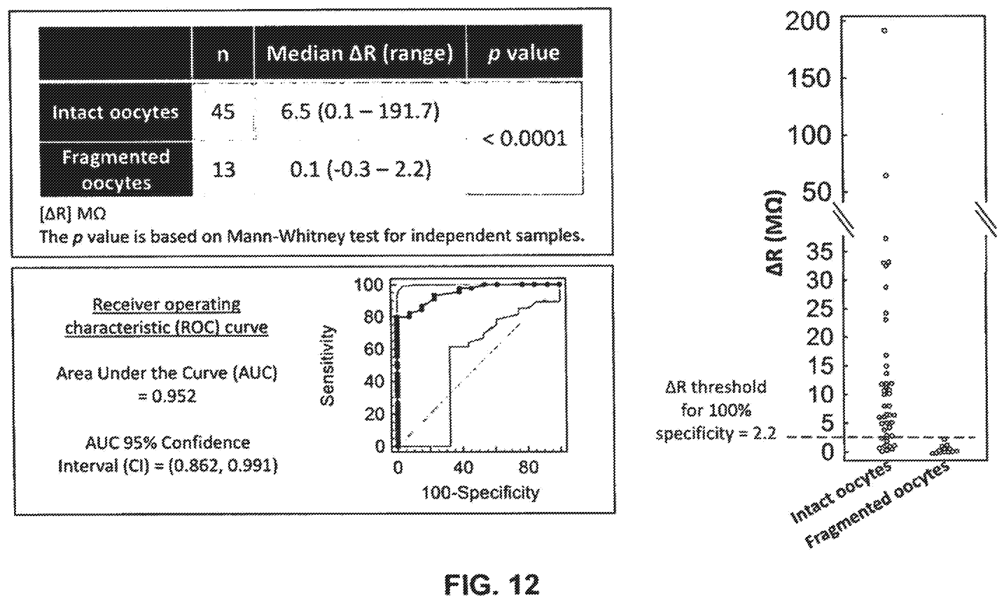

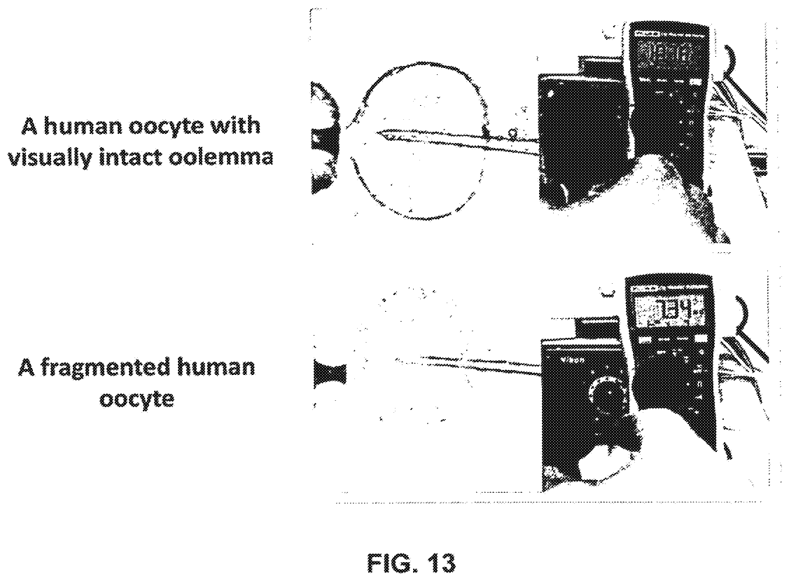

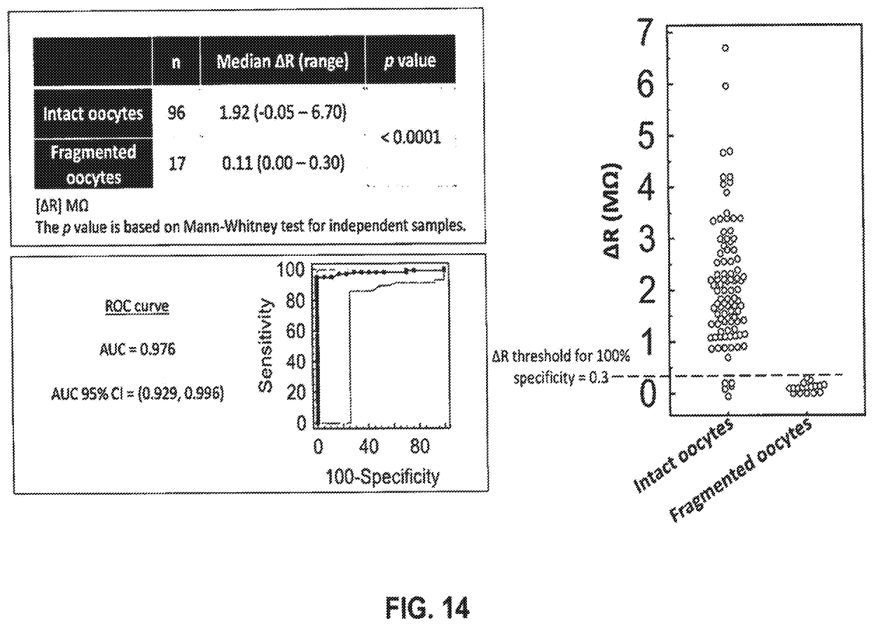

A visual method of assessing membrane piercing was performed in WO 2020/077244 according to a highly accepted method in the field (Mansour R, Intracytoplasmic sperm injection: a state of the art technique. Hum Reprod Update. (1998) 4(1): 43-56. In addition, membrane piercing was confirmed retrospectively by determining cell membrane distention and rupture following the application of positive pressure through the ICSI pipette. If the pipette tip was truly inside the cell, the membrane would rupture following the application of positive pressure through the pipette. If the pipette was outside the cell, only the zona pellucida would distend (FIGS. 8A-8B). While this method provided confirmation of membrane piercing, it did not provide any information about the membrane integrity, and thus could not be used to determine the viability of the cell. Additionally, the application of pressure often killed the cell if the pipette tip was indeed in the cell (compare FIG. 9, showing no penetration of the ICSI pipette before application of positive pressure, to FIG. 10, showing penetration of the ICSI pipette before application of positive pressure).

It was found in WO 2020/077244 that a cell that had a fragmented/ruptured membrane (FIG. 7), as determined by visual assessment, gave little to no increase in resistance when tested with the ICSI pipette (FIG. 3, group n=12).

It was further found that use of visual methods only (standard light microscopy with morphology evaluation) was not sufficient to confirm cell membrane penetration and cell viability. While there were cells that appeared to have normal morphology on visual examination, some of those cells did not show significant increase in resistance upon pipette advancement (ΔR≤2.2 MΩ in this study, 9/45 eggs). Without wishing to be bound by theory, it is suggested that either these cells were not actually penetrated, or the cells were penetrated but their membrane was already damaged. One of the reasons for a decrease in membrane resistance can be the formation of submicroscopic pores in the membrane due to stress. Harvested eggs are under such stress, which can initiate apoptosis or even cause necrosis. Generally, the processes of apoptosis and necrosis generate approximately 10 nm membrane pores, which lead to a decrease in membrane resistance due to lack of its integrity. As light microscopy provides a maximum resolution of 200 nm, these pores are not visible by standard visual light microscopy assessments. In other words, the measured electrical changes often precede light microscopy morphology changes and therefore can detect a dying cell earlier. A third reason for a small resistance increase is a very large leak current around the ICSI pipette. This may be a result of a traumatic entry into the cell that creates a large hole/tear in the membrane.

The zona pellucida (ZP; a specialized extracellular matrix, largely made of glycoproteins, surrounding the developing oocyte within each follicle within the ovary) was also found to have no significant effect on measurements of membrane resistance using the ICSI pipette. Specifically, membrane testing did not show any added significant resistance when touching, going through, or piercing the ZP (FIG. 3, group with n=7 as well as some eggs in other groups and FIGS. 8A-8B).

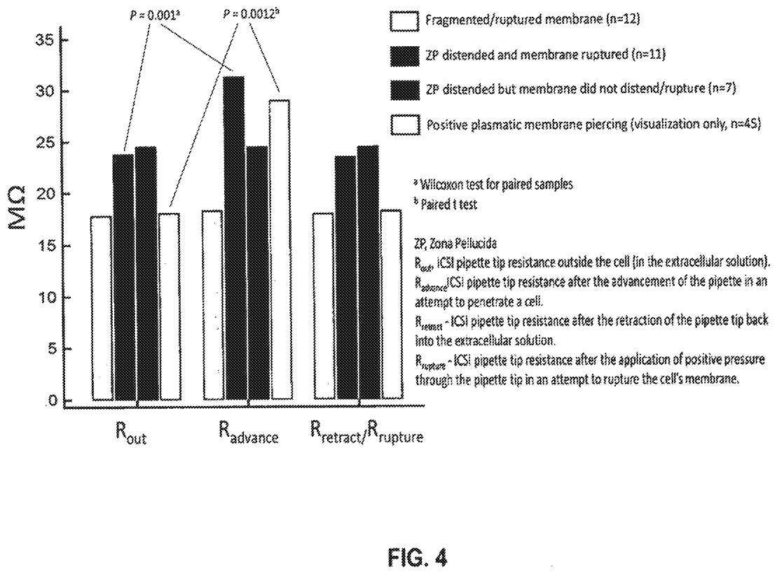

It was also confirmed in WO 2020/077244 that only touching the membrane without penetrating it did not lead to a significant increase in resistance (FIG. 3, compare groups with n=7 vs n=11 and FIGS. 8A-8B). Without wishing to be bound by theory, it is suggested that the use of pipettes with large bore diameters, rather than the standard narrow bore electrophysiology pipettes, allowed a reliable measurement of resistance changes as well as the injection of sperm cells. Of note, the sharp narrow electrophysiology pipettes were more likely to form a “giga seal” (a significant increase in measured pipette tip resistance upon touching the plasmatic membrane) than large bore needle shaped pipettes (the ICSI pipettes). Additionally, unlike the narrow electrophysiology pipettes, sludge in the large bore pipette did not affect its resistance. In none of the 78 cases was the ICSI pipette tip resistance affected by sludge, nor by touching the cell membrane without piercing it (Table 1 and FIG. 4 and FIGS. 8A-8B).

TABLE 1
Resistance measurements of the ICSI pipette tip

| | | | R_{advance} | | R_{retract} | | Rᵣᵤₚₜᵤᵣₑ |
|---|---|---|---|---|---|---|---|
| Group | Rₒᵤₜ (MΩ) | | (MΩ) | | (MΩ) | | (MΩ) |
| Positive plasmatic | 20.3 ± 0.8 | | 35.7 ± 4.6 | | 20.5 ± 0.8 | | — |
| membrane piercing | 18.1 | (15.6-35.9) | 29.0 | (16.3-216.0) | 18.3 | (15.4-36.0) | — |
| determined by | | | | | | | |
| visualization only | | | | | | | |
| (n = 45)ª | | | | | | | |
| Fragmented/ruptured | 17.9 | (15.7-20.2) | 18.2 | (15.4-21.2) | 17.9 | (15.4-20.3) | — |
| membrane (n = 12)^{b} | | | | | | | |

| | | | | | | | |
|---|---|---|---|---|---|---|---|
| Zona Pellucida distended | 23.8 | (17-24.3) | 31.3 | (21.0-130) | — | 23.5 | (17.0-24.6) |
| and membrane ruptured | | | | | | | |
| (n = 11)^{b} | | | | | | | |
| Zona Pellucida distended | 24.5 | (23.9-25.3) | 24.5 | (23.4-25.2) | — | 24.5 | (23.6-25.2) |
| but membrane did not | | | | | | | |
| distend/ rupture (n = 7)^{b} | | | | | | | |

^{a}Data are presented as mean ± standard error of the mean (SEM) in upper row and median with range (in parenthesis in lower row).
^{b}Data are presented as median with range (in parenthesis).
ZP - Zona Pellucida
Rₒᵤₜ - ICSI pipette tip resistance outside the cell (in the extracellular solution).
R_{advance} - ICSI pipette tip resistance after the advancement of the pipette in an attempt to penetrate a cell.
R_{retract} - ICSI pipette tip resistance after the retraction of the pipette tip back into the extracellular solution.
Rᵣᵤₚₜᵤᵣₑ - ICSI pipette tip resistance after the application of positive pressure through the pipette tip in an attempt to rupture the cell's membrane.

Further testing was done in WO 2020/077244 to assess the ability of the visual method to determine cell membrane integrity and thus viability. A ROC curve comparing ΔR in the fragmented/ruptured membrane group (n=12) to AR in the positive plasmatic membrane piercing group (visualization only, n=45) was performed (FIG. 5A, raw data, FIG. 5B, ROC curve). The ROC curve showed that the visual method is not sufficient for a diagnostic test (e.g., membrane integrity and cell viability). More specifically, the ROC curve showed the lower bound of the confidence interval to approach the diagonal linear line, which suggests that visual inspection alone is not sufficient to provide reliable cell viability data or confirm membrane piercing.

### Example 5: In-Vitro Fertilization Experiment

According to WO 2020/077244, three eggs with measured membrane resistance compatible with viable cells (ΔR>2.2 MΩ) were incubated with mouse sperm cells. Within 24 hours two out of the three cells were fertilized showing two-cell embryos. Of note, resting membrane potential was measured in all three eggs and was 0 mV.

The conclusions from this experiment are as follows:
1. Measuring membrane resistance with a large bore pipette is safe and does not lead to cell destruction. The eggs remained alive and kept their fertilization potential after the resistance measurement methods according to the invention.
2. Resting membrane potential in viable cells can be cancelled due to, for example, a leak current around the ICSI pipette. A current that the egg cannot compensate for. Therefore, membrane potential measurement is not a reliable indicator for cell penetration, membrane integrity, or cell viability when using large bore pipettes.
3. The leak current does not interfere with detecting increase in electrical resistance when the egg is penetrated.

An alternative to an electrical wire in the pipette lumen can be a conductive material adhered to the inner pipette wall (e.g., glass pipette-carbon fiber).

Results indicate that the higher the resistance increase the better the egg membrane quality is. The higher the resistance increase the better the egg quality is. It was determined that baseline resistance increase can detect dirty or clogged pipette. In addition, baseline resistance decrease can detect and/or indicate pipette breakage.

While resistance was directly measured and used to evaluate the cells, resistance and conductance and impedance can be used interchangeably to evaluate and report cell viability since conductance and impedance are resistance-sensitive computed values. In addition, resistance can be calculated by a direct measurement of generated current to specific voltage pulse or by direct measurement of generated voltage to specific current pulse.

### Example 6. Resistance Measurements of Human Oocytes

In WO 2020/077244, in order to further validate that measuring resistance provides a high fidelity method for evaluating oocyte cell viability, human oocyte membranes punctured/penetrated using a large bore pipette resistance was measured in order to evaluate viability. Results from these measurements are shown in Table 2.

TABLE 2
Resistance Measurements of human oocytes.

| | | | | | | | Category |
|---|---|---|---|---|---|---|---|
| | | | | | | Delta | (1 = visually |
| # | Experiment date | Egg type | Rₒᵤₜ | Rᵢₙ | R_{retract} | R | alive, 0 = dead) |
| 1 | Jan. 30, 19 | GV | 7.80 | 10.35 | 7.76 | 2.55 | 1 |
| 2 | Feb. 12, 19 | GV | 8.13 | 9.05 | 8.19 | 0.92 | 1 |
| 3 | Feb. 12, 19 | GV | 7.84 | 9.38 | 8.00 | 1.54 | 1 |
| 4 | | GV | 7.70 | 8.80 | 7.78 | 1.1 | 1 |
| 5 | | GV | 9.40 | 11.40 | 9.44 | 2 | 1 |
| 6 | | GV | 8.22 | 9.44 | 8.60 | 1.22 | 1 |
| 7 | | GV | 8.98 | 10.58 | 9.40 | 1.6 | 1 |
| 8 | Feb. 14, 19 | GV | 9.87 | 10.00 | 10.00 | 0.13 | 0 |
| 9 | | GV | 8.55 | 8.55 | 8.50 | 0 | 0 |
| 10 | Feb. 16, 19 | GV | 8.39 | 10.70 | 8.74 | 2.31 | 1 |
| 11 | | GV | 8.45 | 10.64 | 8.53 | 2.19 | 1 |
| 12 | | GV | 8.60 | 12.70 | 8.60 | 4.1 | 1 |
| 13 | Feb. 18, 19 | GV | 7.29 | 10.00 | 7.30 | 2.71 | 1 |
| 14 | | GV | 7.30 | 9.85 | 7.40 | 2.55 | 1 |
| 15 | Feb. 18, 19 | GV | 8.40 | 10.20 | 8.40 | 1.8 | 1 |
| 16 | | GV | 8.30 | 10.68 | 8.40 | 2.38 | 1 |
| 17 | | GV | 8.35 | 10.52 | 7.95 | 2.17 | 1 |
| 18 | Feb. 19, 19 | GV | 10.30 | 12.50 | 10.86 | 2.2 | 1 |
| 19 | | GV | 10.95 | 11.10 | 11.10 | 0.15 | 0 |
| 20 | Feb. 21, 19 | GV | 8.52 | 10.64 | 8.59 | 2.12 | 1 |
| 21 | Feb. 25, 19 | GV | 9.60 | 13.80 | 9.60 | 4.2 | 1 |
| 22 | Feb. 25, 19 | GV | 9.80 | 12.00 | 9.90 | 2.2 | 1 |
| 23 | | GV | 9.50 | 16.20 | 9.73 | 6.7 | 1 |
| 24 | Feb. 27, 19 | GV | 10.12 | 12.90 | 10.20 | 2.78 | 1 |
| 25 | | GV | 10.12 | 12.30 | 10.40 | 2.18 | 1 |
| 26 | Feb. 27, 19 | GV | 15.80 | 20.00 | 16.40 | 4.2 | 1 |
| 27 | Mar. 27, 19 | GV | 18.90 | 21.20 | 18.70 | 2.3 | 1 |
| 28 | | GV | 25.70 | 30.40 | 26.50 | 4.7 | 1 |
| 29 | Mar. 27, 19 | GV | 24.20 | 24.50 | 24.50 | 0.3 | 0 |
| 30 | Mar. 27, 19 | GV | 8.90 | 9.16 | 8.90 | 0.26 | 0 |
| 31 | Apr. 10, 19 | MI | 7.45 | 9.70 | 7.49 | 2.25 | 1 |
| 32 | | MI | 7.48 | 9.70 | 7.47 | 2.22 | 1 |
| 33 | Apr. 11, 19 | Fragmented | 7.63 | 7.8 | 7.65 | 0.17 | 0 |
| 34 | Apr. 16, 19 | MII | 7.55 | 9.73 | 7.64 | 2.18 | 1 |
| 35 | Apr. 18, 19 | GV | 7.98 | 8.85 | 8.13 | 0.87 | 1 |
| 36 | Apr. 22, 19 | GV | 9.50 | 11.60 | 9.50 | 2.1 | 1 |
| 37 | | GV | 7.90 | 8.60 | 7.90 | 0.7 | 1 |
| 38 | | MI | 7.90 | 8.80 | 7.80 | 0.9 | 1 |
| 39 | Apr. 25, 19 | GV | 7.20 | 10.70 | 7.30 | 3.5 | 1 |
| 40 | | GV | 7.57 | 7.77 | 7.69 | 0.2 | 0 |
| 41 | Apr. 24, 19 | MI | 7.91 | 11.30 | 8.06 | 3.39 | 1 |
| 42 | Apr. 24, 19 | MI | 7.30 | 9.90 | 7.70 | 2.6 | 1 |
| 43 | | MI | 7.66 | 9.20 | 7.80 | 1.54 | 1 |
| 44 | | GV | 7.68 | 9.15 | 7.80 | 1.47 | 1 |
| 45 | | GV | 7.74 | 8.83 | 7.80 | 1.09 | 1 |
| 46 | Apr. 24, 19 | MI | 7.34 | 11.40 | 7.85 | 4.06 | 1 |
| 47 | Apr. 25, 19 | MI | 7.70 | 9.05 | 8.04 | 1.35 | 1 |
| 48 | Apr. 30, 19 | GV | 7.82 | 9.60 | 8.14 | 1.78 | 1 |
| 49 | | GV | 8.12 | 9.80 | 8.04 | 1.68 | 1 |
| 50 | | MI | 7.98 | 9.40 | 8.10 | 1.42 | 1 |
| 51 | | MI | 7.93 | 12.60 | 8.00 | 4.67 | 1 |
| 52 | | GV | 7.96 | 9.80 | 8.03 | 1.84 | 1 |
| 53 | | GV | 7.60 | 9.30 | 7.85 | 1.7 | 1 |
| 54 | | GV | 7.84 | 10.70 | 7.96 | 2.86 | 1 |
| 55 | | GV | 7.86 | 10.85 | 7.87 | 2.99 | 1 |
| 56 | | GV | 7.82 | 10.60 | 8.00 | 2.78 | 1 |
| 57 | | GV | 7.94 | 9.34 | 7.81 | 1.4 | 1 |
| 58 | | GV | 7.80 | 9.25 | 7.95 | 1.45 | 1 |
| 59 | | GV | 7.84 | 13.80 | 8.10 | 5.96 | 1 |
| 60 | | GV | 7.90 | 9.30 | 8.05 | 1.4 | 1 |
| 61 | | GV | 7.85 | 9.60 | 7.95 | 1.75 | 1 |
| 62 | | GV | 7.84 | 7.93 | 7.82 | 0.09 | 1 |
| 63 | | GV | 7.80 | 10.80 | 7.93 | 3 | 1 |
| 64 | May 10, 19 | MI | 8.10 | 10.30 | 8.00 | 2.2 | 1 |
| 65 | May 13, 19 | GV | 7.09 | 9.40 | 7.40 | 2.31 | 1 |
| 66 | | MI | 7.17 | 9.70 | 7.20 | 2.53 | 1 |
| 67 | | MI | 7.30 | 8.65 | 7.30 | 1.35 | 1 |
| 68 | May 13, 19 | GV | 7.26 | 8.40 | 7.30 | 1.14 | 1 |
| 69 | | GV | 7.32 | 8.40 | 7.35 | 1.08 | 1 |
| 70 | | MI | 7.26 | 8.15 | 7.23 | 0.89 | 1 |
| 71 | | GV | 7.22 | 8.70 | 7.50 | 1.48 | 1 |
| 72 | | GV | 7.26 | 10.40 | 7.30 | 3.14 | 1 |
| 73 | | Atretic | 7.00 | 7.14 | 7.11 | 0.14 | 0 |
| 74 | | GV | 7.22 | 8.10 | 7.35 | 0.88 | 1 |
| 75 | | GV | 7.29 | 8.40 | 7.45 | 1.11 | 1 |
| 76 | | GV | 7.35 | 8.50 | 7.00 | 1.15 | 1 |
| 77 | | GV | 7.40 | 8.65 | 7.40 | 1.25 | 1 |
| 78 | May 14, 19 | GV | 7.26 | 8.38 | 7.40 | 1.12 | 1 |
| 79 | | GV | 7.65 | 9.25 | 7.50 | 1.6 | 1 |
| 80 | | MI | 6.75 | 8.40 | 7.10 | 1.65 | 1 |
| 81 | | GV | 7.12 | 8.95 | 7.30 | 1.83 | 1 |
| 82 | May 19, 19 | MII | 15.40 | 16.80 | 15.40 | 1.4 | 1 |
| 83 | May 30, 19 | MI | 7.90 | 9.00 | 7.90 | 1.1 | 1 |
| 84 | | MI | 7.71 | 9.70 | 7.68 | 1.99 | 1 |
| 85 | Jun. 3, 19 | GV | 9.04 | 12.20 | 9.74 | 3.16 | 1 |
| 86 | | MI | 8.57 | 8.52 | 8.55 | −0.05 | 1 |
| 87 | | GV | 8.25 | 8.25 | 8.23 | 0 | 0 |
| 88 | | MI | 8.18 | 10.20 | 8.25 | 2.02 | 1 |
| 89 | | GV | 8.24 | 8.26 | 8.24 | 0.02 | 0 |
| 90 | | GV | 8.17 | 8.27 | 8.19 | 0.1 | 0 |
| 91 | Jun. 12, 19 | GV | 11.5 | 13.2 | 11.7 | 1.7 | 1 |
| 92 | Jun. 13, 19 | MI | 9.90 | 11.10 | 10.06 | 1.2 | 1 |
| 93 | | MI | 10.22 | 11.10 | 10.24 | 0.88 | 1 |
| 94 | | MI | 10.26 | 12.00 | 10.30 | 1.74 | 1 |
| 95 | Jun. 14, 19 | GV | 13.90 | 17.30 | 14.30 | 3.4 | 1 |
| 96 | Jun. 19, 19 | GV | 11.00 | 12.70 | 11.20 | 1.7 | 1 |
| 97 | | Atretic | 10.60 | 10.70 | 10.70 | 0.1 | 0 |
| 98 | Jun. 24, 19 | MI | 10.15 | 12 | 9.9 | 1.85 | 1 |
| 99 | | GV | 10.3 | 13.65 | 10.25 | 3.35 | 1 |
| 100 | | Fragmented | 10.28 | 10.4 | 10.24 | 0.12 | 0 |
| 101 | | GV | 10.11 | 13.1 | 10.05 | 2.99 | 1 |
| 102 | | GV | 9.84 | 9.95 | 9.85 | 0.11 | 0 |
| 103 | | MI | 10.1 | 14 | 10.16 | 3.9 | 1 |
| 104 | | GV | 10.6 | 10.6 | 10.46 | 0 | 0 |
| 105 | Jun. 24, 19 | MI | 10.36 | 13.3 | 10.2 | 2.94 | 1 |
| 106 | Jun. 27, 19 | MI | 10.15 | 10.3 | 10.13 | 0.15 | 1 |
| 107 | | Atretic | 9.7 | 9.8 | 9.6 | 0.1 | 0 |
| 108 | | GV | 9.5 | 9.7 | 9.5 | 0.2 | 1 |
| 109 | | GV | 10 | 10.2 | 10.1 | 0.2 | 1 |
| 110 | | GV | 10 | 13.4 | 10.4 | 3.4 | 1 |
| 111 | | GV | 10.4 | 12.4 | 10.4 | 2 | 1 |
| 112 | | GV | 10.5 | 10.5 | 10.4 | 0 | 0 |
| 113 | | GV | 10.6 | 14 | 10.8 | 3.4 | 1 |

Human eggs were tested as well. Results indicate that human eggs showed lower resistance increase in comparison to mouse eggs. A resistance increase of at least 0.3 MΩ was consistent with a detection of a viable human egg. The reason for this may be the larger size of human eggs (i.e., a larger membrane surface area). Results in all cells experiments also indicate that the zona pellucida does not significantly affect the measured resistance.

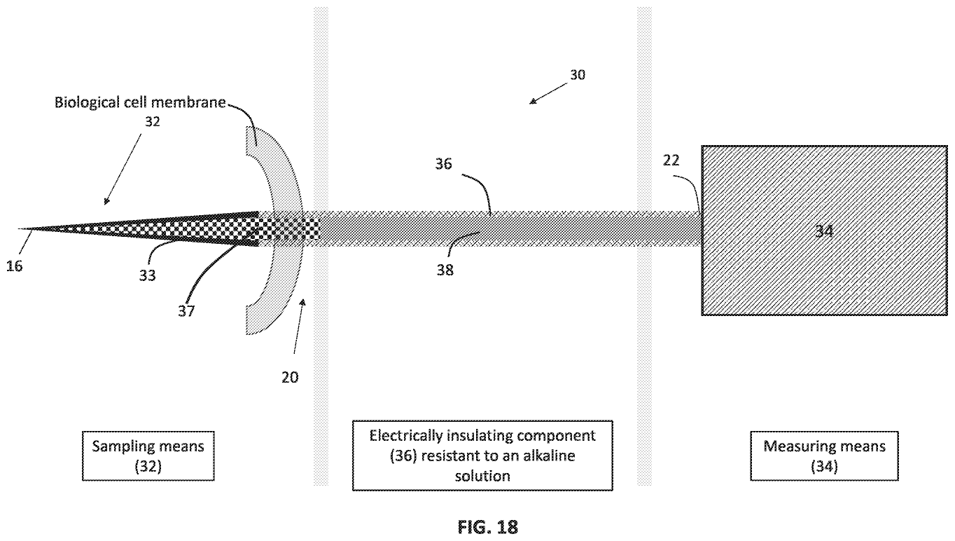

Referring now to FIG. 18, there is shown an overview of an embodiment of a detection apparatus (30) in accordance with the principles of the present invention, comprising a sampling means (32), a measuring means (34), and an electrically insulating component (36) located between sampling means (32) and measuring means (34). An electrically conductive material (38) is provided within electrically insulating component (36). Electrically insulating component (36) has resistance to an alkaline solution of pH 8 to 11, wherein said resistance to said alkaline solution comprises at least one property selected from the group consisting of resistance to formation of microcracks for at least 1 minute upon exposure to said alkaline solution; resistance to electrical insulation failure for at least 12 minutes upon exposure to said alkaline solution; and resistance to visible structural integrity failure for at least 20 minutes upon exposure to said alkaline solution. The sampling means (32) comprises at least a portion configured to be inserted into a biological cell by piercing its plasmatic membrane.

According to an exemplary embodiment, detection apparatus (30) comprises an electrically conductive wire (20) having a first end (16) and a second end (22), wherein a first section of wire (20) constitutes sampling means (20) and a second section of wire (20) constitutes electrically conductive material (38). During use, first end (16) of wire (20) is inserted within an electrolytic solution, such as an extracellular or intracellular solution, while measuring means (34), such as an electrical resistance meter or an electrical capacitance meter is connected to second end (22) of wire (20).

In some embodiments, an entire length of wire (20) is formed from a same material, such as comprising silver, copper or gold, or combinations thereof.

In an alternative embodiment, wire (20) a first section of wire (20) which constitutes sampling means (32) is formed from a first material, such as silver; while a second section of wire (20) which constitutes electrically conductive material (38) may be formed from a different material, which may be any suitable electrically conductive material, including but not limited to an electrically conductive metal other than silver, e.g. copper, gold; an electrically conductive non-metal material (such as graphite); and an electrolytic fluid (e.g., a liquid or a gel). Two types of electrically conductive metal wires can be welded together or connected through male-female plugs.

According to an embodiment, as shown in FIG. 18, sampling means (32) comprises a silver metal wire (20) wherein end (16) has a sharp tip and is coated with chloride (33). The chloride coating is optional and is used for obtaining more stable electrical reading.

Electrically insulating component (36) is provided as a coating layer along a portion of wire (20), such that a first end comprising tip (16) and a second end (22) are exposed.

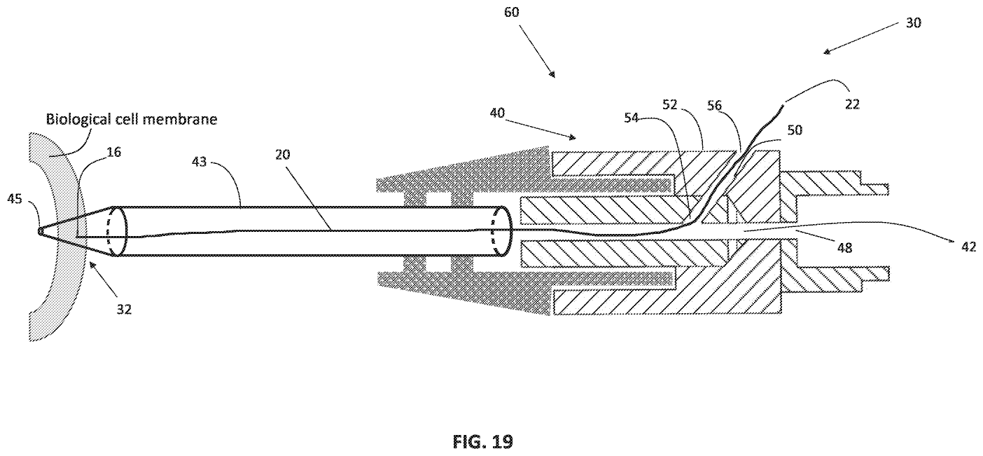

According to an alternative embodiment, as shown in FIG. 19, sampling means (32) comprises a micropipette (43) having a sharp tip (45) (made from glass, plastic, carbon fiber, silicon, etc.) filled with an electrolyte. In this embodiment, electrically conductive material (38) comprises chloride coated (partially or fully) silver wire (20). During use, at least a portion of wire (20) is inserted into an electrolytic solution provided in micropipette (43).

According to an alternative embodiment, the sampling means is only a metal wire tip or a micropipette tip.

As a non-limiting example, electrically conductive material in the sampling means (32) comprises silver (37). In order to obtain stable electrical readings in aqueous solutions, it is necessary to coat the silver (37) with chloride (33). This may be achieved by exposing electrically insulating component (36), containing therein electrically conductive material (38), to bleach, such as by positioning electrically insulating component (36) over tubes containing bleach or immersing electrically insulating component (36) in a solution containing bleach. End (16) of wire (20) which protrude from electrically insulating component (36) is exposed to the bleach solution, until an advanced stage of the coating process is identified by the color of wire (20) turning black.

According to an embodiment, the conductive fluid may comprise a mineral oil mixed with a salt such as salt of an alkali metal or alkaline earth metal (for example, a salt selected from the group consisting of sodium chloride, potassium chloride, magnesium chloride, sodium sulfate and combinations thereof), optionally further comprising a detergent (such as a salt of a fatty acid). The mineral oil may comprise a synthetic oil (such as an oil selected from the group consisting of TriboSys™, polyalphaolefins, diesters, polyolesters, alkylated benzenes, phosphate esters and combinations thereof); or a natural oil (such as an oil selected from the group consisting of canola oil, castor oil, palm oil and combinations thereof).

According to an embodiment the electrically conductive comprises grease harboring high silver content.

According to an embodiment, a metal plate is provided (flush with a lumen provided within the electrically insulating component) which is in contact with the mixture of the mineral oil and salt. According to an alternative embodiment, the mixture of the mineral oil and salt is provided within the sampling means only, and a metal wire extends outwards from the electrically insulating means to contact the mixture of oil and salt within the sampling means.

According to an embodiment, the conductive fluid (including a gas) can be formed from materials which do not necessarily include any one of an oil, a salt, or a detergent. A non-limiting example of such a material is mercury.

Prior art insulating components for cell assessment systems are formed from polycarbonate, due to its desirable physical and chemical properties, such as high strength and ease of molding.

The present inventor noted problems associated with prior art systems, such as poor pressure/sealing of electrically insulating components. This was surprisingly identified as occurring following exposure of the polycarbonate to bleach vapor.

Therefore, according to the principles of the present invention, in order to avoid structure failures, the electrically insulating material surrounding the silver must be bleach resistant (i.e., resistant to alkaline solutions containing chlorine ions).

Examples of suitable alkaline-resistant materials include, without limitation, polyvinyl chloride (PVC), glass, silicone, polyethylene, polypropylene and polyurethane, or combinations thereof.

The sampling means can sense electrical signals outside the cell (when placed in the extracellular solution), form the cell's membrane (if in contact or close proximity with the membrane), or from the intracellular solution (if fully inserted into the cell).

The measuring means can be a handheld portable multimeter, and electrical resistance meter, an electrical capacitance meter, an electrical voltage meter, an electrophysiology amplifier, etc.

These components form the detection apparatus that is capable of providing data about cell viability and plasmatic membrane piercing. These data are crucial, for example, for selecting viable oocytes for sperm injection.

In some embodiments, as shown in FIG. 19, electrically insulating component (36) is provided as a connector (also referred to herein as an adapter) (60) which further serves to integrate sampling means (32) with measuring means (34) and optionally further with an injection system. The portions of connector (60) which during use are in contact with fluid of an injection system (such as aqueous solutions, air or oil) have resistance to an alkaline solution of pH 8 to 11.

Electrically conductive material (38) provides an electrical connection between an electrolytic solution within injection device (2) and measuring means (34). Electrically conductive material (38) may comprise a wire (20) extending between sampling means (32) and measuring means (34).

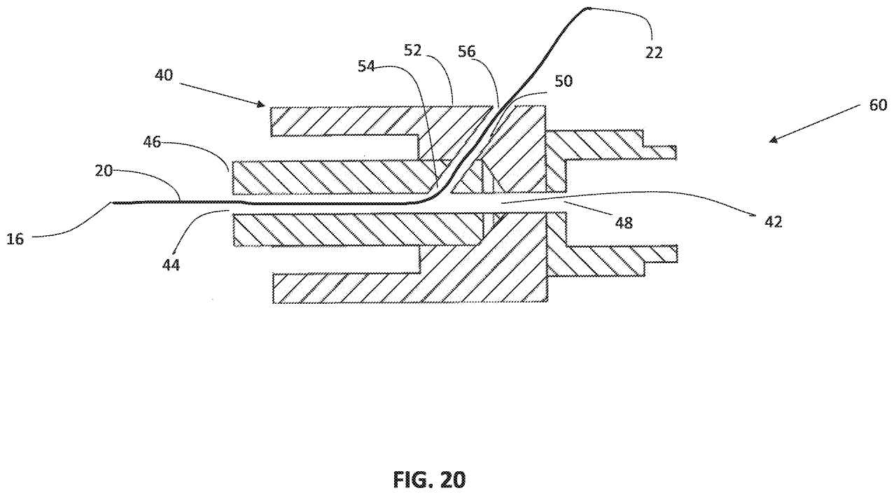

As shown in FIG. 20, according to an embodiment, adaptor (60) comprises a profile (40), having a first internal channel (42) provided along a longitudinal axis of the adaptor, first channel (42) having a first opening (44) at a first end (46) of the adaptor and a second opening (48) at a second end (49) of the adaptor. Channel (42) is adapted to receive therein fluid.

Adaptor (60) further comprises a second channel (50) having a first opening (54) at an intersection point (51) within first channel (42) and a second opening (56) provided through external surface (52) of adaptor (60). Adaptor (60) is configured to receive at least a portion of wire (20) extending outwards from first opening (44) of first channel (42) and second opening (56) of second channel (50).

Adaptor (60) further comprises sealing material(s) which maintain positive and negative pressure of air, water, or oil during the fluid micromanipulation and cell microinjection processes.

Wire (20) is inserted into adaptor (60) through first opening (56) of second channel (50) and out through second opening (44) of first channel (42) such that first end (16) of wire (20) extends out of opening (44) and second end (22) of wire (20) extends out of opening (56). First end (16) of wire (20) is inserted into electrolytic solution (18) contained within injection pipette (2) and second end (22) of wire (20) is connected to meter (17). Alternatively, wire (20) may be replaced by conductive material (38) attached in a continuous arrangement to a wall of first channel (42) from first opening (44) to intersection point (51) and further to second opening (56) of second channel (50).

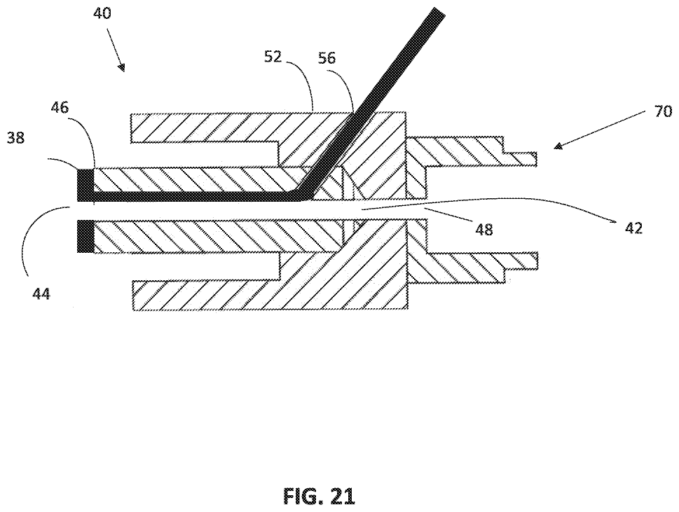

Alternatively, as shown in FIG. 21, there is provided an adaptor (70) wherein wire (20) of FIG. 20 is replaced by an electrically conductive material (38) provided as a coating along the path of the channel defined above for wire (20). In such an embodiment, electrically conductive material (38) may further be provided along ends (46) of adaptor (70).

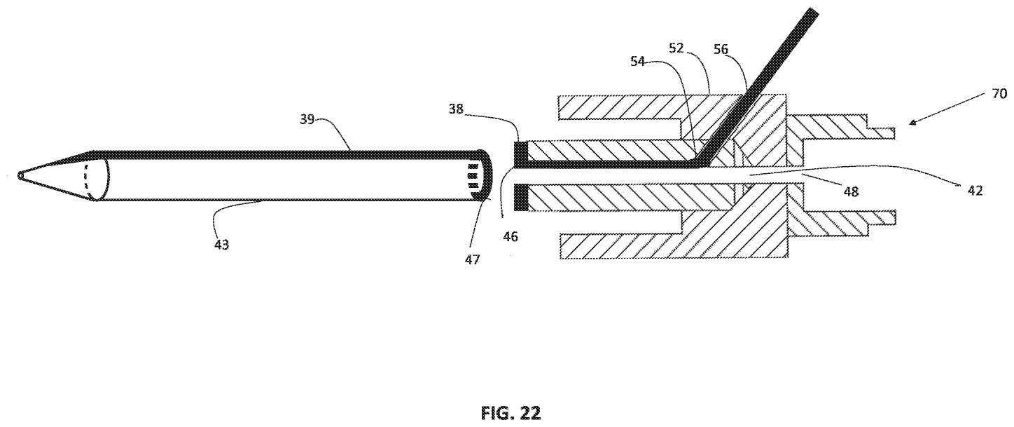

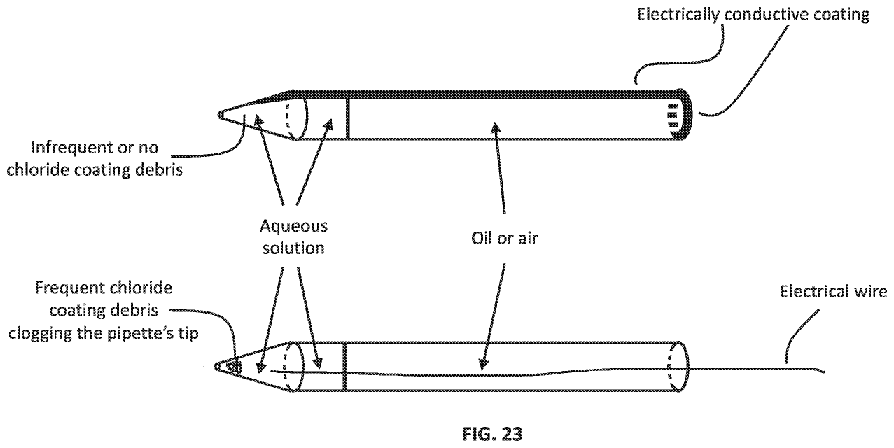

FIG. 22 shows the adaptor (70) of FIG. 21 in combination with a sampling means (32) comprising a micropipette (43), wherein electrically conductive material (39) is provided as a coating along an inner wall of micropipette (43) and around a distal end (47). Use of a coating layer of electrically conductive material instead of a wire, has a number of advantages, such as infrequent or no chloride-coating debris, in contrast to the significant amounts of such debris which is found with use of a wire as the electrically conductive material, resulting in frequent blockage of the narrow pipette tip opening (FIG. 23). The presence of such debris may cause clogging of the tip of the micropipette. This frequent clogging required frequent pipette replacements. Furthermore, the electrically conductive coating does not interfere with flow of fluid through the pipette tip during use. Another advantage is that the conductive coating does not interfere with flow of the fluid in the pipette itself and in the adapter. FIG. 23 demonstrates these problems and their solution by using a conductible material attached to the pipette's wall.

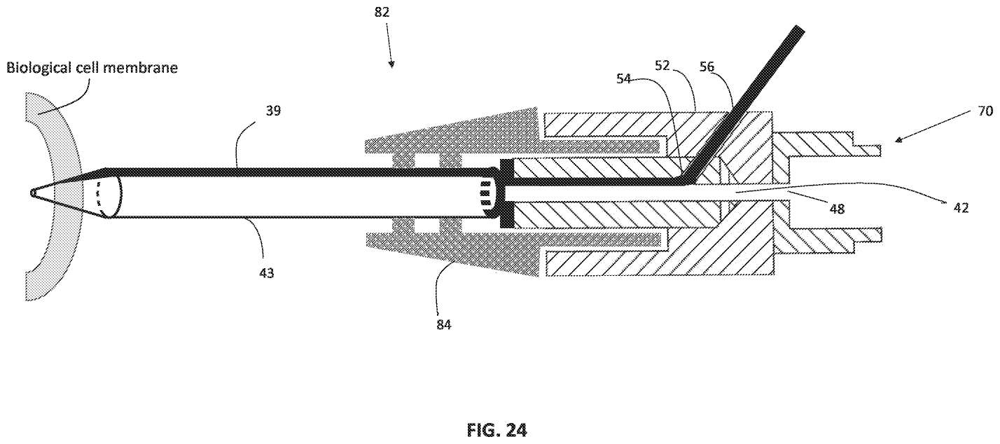

FIG. 24 shows a system (82) comprising adaptor (70) and micropipette (43) of FIG. 22, and further comprising a pipette grip (84) which holds end (46) of adaptor (70) in contact with distal end (47) of micropipette (43).

### Example 7: Investigation of the Failure of a Polycarbonate Adapter Following Exposure to Bleach

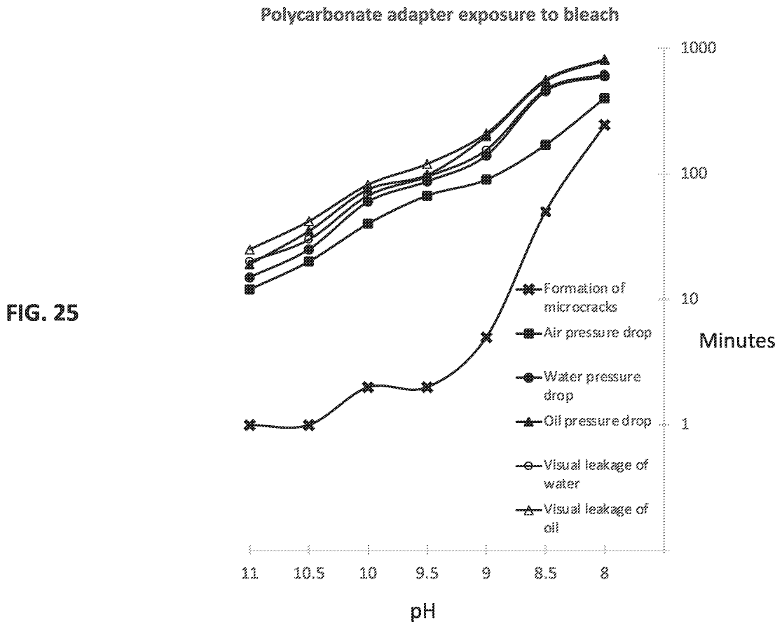

Bleach solutions have a pH of 11. Prior art polycarbonate adapters were exposed to concentrated and diluted bleach solutions (pH range 8-11). FIG. 25 demonstrates the failure of the structure of the adaptor and sealing loss over time.

The sealing ability loss of the polycarbonate adapter was evaluated for leakage of the following fluids which are commonly used in in vitro fertilization (IVF) intracytoplasmic sperm injection (ICSI) procedures:
1. mineral oil
2. water
3. air

The seal loss was evaluated in 3 ways:
1. Formation of microcracks.
2. Fluid (air, water, or oil) pressure drop and/or electrical insulation loss.
3. Visual leakage of fluid (water or oil) through the adapter's wall(s) (i.e., visual structural failure).

Prior art adapter made of polycarbonate was exposed to various concentrations of bleach solution (pH of 8 to 11). The time taken (in minutes) to formation of microcracks, fluid pressure drops, and visual leakage of fluid was determined. Seal loss was found to occur faster on exposure to a stronger alkaline environment (higher pH, FIG. 25).

Alkaline-resistant adaptors made of polyvinyl chloride (PVC) plastic or glass in accordance with the principles of the present invention, were made. It was found that such alkaline-resistant adaptors did not lose their sealing capabilities when exposed to an alkaline environment of pH 8-11. The adaptors maintained their structural properties even when submerged in bleaching solutions (pH range 8-11) for at least 3 minutes and up to 24 hours.

It was concluded that a weak base such as diluted bleach attacks and leads to structural integrity loss of polycarbonate plastic whereas PVC and glass are resistant to bleach attack (i.e., no microcracks or seal/insulation loss) at least under the experimental conditions used. Table 3 summarizes the experimental results relating to exposure of different materials to bleach solutions at various pH values.

Polycarbonate is generally considered to be a preferred material over PVC or glass when making an adapter (i.e., an electrically insulating component) in an IVF/ICSI/cell injection system for the following reasons:
1. Polycarbonate is an FDA approved biocompatible material.
2. PVC is considered toxic.
3. Polycarbonate avoids breakage that can create an unsafe environment in a wide variety of items such as tubes and connectors. Therefore, medical devices that were once made of glass are now made of polycarbonate.

However, the present inventors have surprisingly found that bleach damages polycarbonate adapters and have proposed various biocompatible materials (other than PVC and glass) as alternatives to polycarbonate in the preparation of such an adaptor (see Table 3).

TABLE 3
Resistance of selected materials to bleach solutions at various pH values.

| | | | Stainless | | | | | |
|---|---|---|---|---|---|---|---|---|
| | Polycarbonate | PVC | Steel | Glass | Silicone | Polyethylene | Polypropylene | Polyurethane |
| PH 8 | Limited life | Resistant | Limited | Resistant | Resistant | Resistant | Resistant | Resistant |
| | | | life | | | | | |
| pH 9 | Sensitive | Resistant | Limited | Resistant | Resistant | Resistant | Resistant | Resistant |
| | | | life | | | | | |
| PH | Sensitive | Resistant | Limited | Resistant | Resistant | Resistant | Resistant | Resistant |
| 10 | | | life | | | | | |
| pH | Sensitive | Resistant | Limited | Resistant | Resistant | Resistant | Resistant | Resistant |
| 11 | | | life | | | | | |

### Example 8: Overcoming Micropipette Clogging

To achieve fine fluid micromanipulation through the pipettes, usually only their tips are filled with aqueous solutions. The majority of the pipette's lumen contains oil or air. The silver wires (or other conductive materials/structures) have to pass through the oil or air phases (electrical insulators) and reach the aqueous phase in the micropipettes (the electrically conductive phase, FIG. 23).

Surprisingly, the silver wires (coated with chloride) were shedding the chloride coating during the electrical measurements as well as during the manipulation of the fluid flow through the holding and injection micropipettes. This resulted in frequent and unexpected clogging of the micropipettes' tips (by coating debris) and required their frequent replacement by new ones. Additionally, the silver wire itself, sitting in the micropipettes, interfered with the fine and precise fluid micromanipulation. When ICSI was attempted, there were multiple challenges delivering the sperm safely and securely in the oocyte. When RNA/DNA/proteins were injected into cell through much smaller pipette tips (less than 1 micron), the challenges were significant as well.

To overcome these issues, the inventor created custom pipettes. These custom micropipettes contained fine silver layer applied to their inner wall and back end (the connection point to the adapters). This silver layer covered the wall partially to keep the micropipette glass wall transparent (FIGS. 22-24).

The fine silver layer was then coated with chloride. The methods to do so were flushing bleach through the pipettes' lumina or passing electrical current through the silver layer while it is in contact with chloride containing solution.

The adapters (allowing the connection to an electrical amplifier/resistance meter) were designed to have a physical connection of a conductive material with the micropipette's back end (which was electrically conductive as well, FIGS. 21, 22 and 24).

This design eliminated the need to place electrical wires in solutions and therefore, the chloride coating shedding was significantly reduced or eliminated. Additionally, the inventor was able to gain better control of the fluid micromanipulation in and out of the injection and holding micropipettes (since there was no bulky electrical wire sitting in the solution in the micropipettes). This design allowed the inventor to obtain stable electrical recordings without frequent replacement of the pipettes. With this improved design the inventor was able to determine (in real time) cell viability and plasmatic membrane piercing and to perform cell microinjection of DNA/RNA/proteins as well as ICSI procedure safely and securely.

### Example 9: Electrically Conductive Oil as Electrically Conductive Material in a Micropipette

A detection apparatus as disclosed herein is provided, comprising a measuring means; a sampling means comprising a glass micropipette; and an electrically insulating component comprising a plastic adaptor located between the measuring means and the sampling means. The adaptor comprises a lumen, containing therein a silver wire, which extends out of the adaptor on one side to connect to the measuring means (e.g., an electrophysiology amplifier or a multimeter).

Option I—Mineral oil is mixed with NaCl and a detergent to serve as an electrically conductive material within the lumen of the sampling means comprising a glass micropipette. The glass micropipette is filled with the mixture of mineral oil and NaCl. The mixture is then allowed to flow into the lumen of the adaptor where it contacts the silver wire. The silver wire extends out of the adapter on one side and is connected to the measuring means. In such way the present inventor is able to avoid inserting a silver wire into the glass micropipette sampling component, while using mineral oil which is commonly used by embryologists while performing ICSI. Option II—As an alternative to the silver wire provided as an electrically conductive material within the lumen of the electrically insulating component of FIG. 19 or 20, a silver plate is inserted, flush with the wall of the lumen of the electrically insulating component. In such an embodiment, the silver plate is in contact with the electrically conductive oil. By creating this embodiment, the present inventor was able to overcome the interference to oil flow (through the lumen of the electrically insulating component and/or of the micropipette as sampling means) caused by the silver wire.

### Example 10: Egg Activation and Increasing Fertilization Rate

Sperm entering (or injected into) an egg should naturally activate the egg and lead to fertilization and normal cell divisions (the creation of an embryo). There are several medical conditions in which sperm in not able to properly activate an egg (e.g., globozoospermia) or the egg is not able to correctly sense appropriate chemical/electrical signals from the sperm. To date, oocyte electro-activation has been done with electrodes located extracellularly and by exposure to high voltages (which may damage the egg/sperm). An alternative and safer approach can be a lower voltage exposure (within the oocyte's physiological range) during ICSI while the ICSI pipette tip is in the egg. Egg activation, previously done before or after ICSI outside the cell, can now be performed from within the cell using the technique described in this invention.

An embodiment of the system described herein can activate an egg through the generation of electrical pulses through an injection pipette (outside the egg, in contact with the egg's membrane, or from within the egg). In this manner the present inventor was able to activate eggs and generate parthenogenesis. More specifically, the inventor compared traditional ICSI (no voltage application, 100 mouse eggs) to ICSI with voltage application though the ICSI micropipette (100 mouse eggs): the present inventor was able to increase mouse egg fertilization rate (from 20% to 63%) when applying near physiological voltage pulses (100 mV 1 mSec square pulse at 50 Hz for 0.5 Sec) during the ICSI procedure (when the micropipette tip was inside the egg). The day 5 embryo (blastocyst stage) yield remained the same (around 50%) in both study arms. This means that egg activation through the ICSI micropipette from within the egg is non-toxic (i.e., not resulting in lower day 5 embryo survival). The voltage pulses used herein are significantly lower and more physiological than voltages traditionally applied to eggs externally (e.g., 2.8 kv/cm) and therefore are safer. This tool can serve as a useful, efficient, and safer treatment option for couples who have failed fertilization (or poor fertilization rate) due to egg or sperm defects such as globozoospermia). Lastly, different electrical voltages and/or currents may be applied to further increase fertilization and embryo survival rates. The stimulation protocol described above is a non-limiting example.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A detection apparatus for testing biological cells, the apparatus comprising
a measuring means for measuring an electrical property of a single biological cell present in a solution; and
sampling means for sampling said solution, a membrane of the cell, and/or an intracellular fluid of the cell; and
an electrically insulating component located between said sampling means and said measuring means, containing therein an electrically conductive material,
wherein said electrically insulating component has resistance to an alkaline solution of pH 8 to 11,
wherein said resistance to said alkaline solution comprises at least one property selected from the group consisting of resistance to formation of microcracks for at least 1 minute upon exposure to said alkaline solution; resistance to electrical insulation failure for at least 12 minutes upon exposure to said alkaline solution; and resistance to visible structural integrity failure for at least 20 minutes upon exposure to said alkaline solution.

2. The detection apparatus of claim 1, wherein said electrical property is selected from the group consisting of (i) membrane potential, wherein said measuring means comprises an electrical potential meter; (ii) membrane resistance, wherein said measuring means comprises an electrical resistance meter; and (iii) membrane capacitance, wherein said measuring means comprises an electrical capacitance meter; or combinations thereof.

3. The detection apparatus of claim 1, wherein the electrically conductive material comprises a metal and/or a conductive fluid.

4. The detection apparatus of claim 3, wherein the conductive material comprises silver coated with chloride.

5. The detection apparatus of claim 3, wherein said electrically conductive material comprises a mineral oil.

6. The detection apparatus of claim 5, further comprising at least one selected from the group consisting of a salt, a detergent and combinations thereof.

7. The detection apparatus of claim 1, wherein the sampling means comprises a micropipette tip or an electrical wire tip.

8. The detection apparatus of claim 1, which is a cell injection system.

9. A system or kit comprising the detection apparatus of claim 1.

10. The system or kit of claim 7, further comprising a holding pipette.

11. A method for selecting a cell for injection of a substance, comprising use of the detection apparatus of claim 1.

12. The method of claim 9, wherein said selecting comprises selecting said cell for injection of a substance thereinto.

13. The method of claim 9, wherein said detection apparatus is computerized and/or automated.

14. A method for selecting a viable cell, comprising use of the apparatus of claim 1.

15. A method of determining viability of a cell, the method comprising:
(a) providing the detection apparatus of claim 1, said sampling means having a tip, wherein said tip is configured to penetrate a membrane of the cell and wherein said measuring means comprises an electrical resistance meter connected directly or indirectly to said tip;
(b) measuring electrical resistance of said tip outside the cell;
(c) inserting said tip into the cell and measuring electrical resistance of said tip inside the cell;
(d) calculating resistance of the cell by subtracting the electrical resistance outside the cell of step (b) from the electrical resistance inside the cell of step (c), and
(e) determining viability of the cell by comparing the resistance of the cell of step (d) with a control;

16. A method for identifying piercing of a cell's membrane by the sampling means, comprising use of the detection apparatus of claim 1.

17. A method for identifying piercing of a cell's membrane, the method comprising:
(a) providing the detection apparatus of claim 1, said sampling means having a tip, wherein said tip is configured for cell penetration, and wherein said measuring means comprises an electrical resistance meter connected directly or indirectly to said tip;
(b) measuring electrical resistance of said tip outside the cell;
(c) advancing said tip towards the cell while measuring electrical resistance of said tip; and
(d) determining membrane piercing by a detected increase in resistance.

18. An injection micropipette having an electrically conductive material on its inner wall.

19. The injection micropipette of claim 18, wherein the electrically conductive material on its inner wall is fine silver coating.

20. The injection micropipette of claim 18, wherein the electrically conductive material on its inner wall is thin silver layer coated with chloride.

21. A cell microinjection system comprising the injection micropipette of claim 18.

22. The cell microinjection system of claim 21, wherein the injection micropipette in claim 16 is connected to an electrical resistance meter.
